(19) United States
(12) Patent Application Publication
(10) Pub. No.: US 2026/0365993 A1
(43) Pub. Date: Nov. 19, 2026

(54) REGULATORY SEQUENCES AND USES THEREOF

(71) Applicant: Legend Biotech USA Inc., Somerset, NJ (US)

(72) Inventors: Yun ZHANG, Nanjing (CN); Junjie LI, Nanjing (CN); Kun JIANG, Nanjing (CN); Jiajia LI, Nanjing (CN); Lewen MA, Nanjing (CN); Yafang LI, Nanjing (CN); Hong LUO, Nanjing (CN); Tailan ZHAN, Nanjing (CN); Tao ZHAO, Nanjing (CN)

(21) Appl. No.: 19/126,525

(22) PCT Filed: Jan. 10, 2024
(86) PCT No.: PCT/CN2024/071518

§ 371 Date: May 1, 2025

(30) Foreign Application Priority Data
Jan. 10, 2023 (WO) PCT/CN2023/071666

(51) Int. Cl. C12N 7/00; A61K 35/17; A61K 40/11; A61K 40/31; A61K 40/32; C07K 14/47; C12N 5/0783; C12N 15/86

(52) U.S. Cl. CPC C12N 7/00; A61K 35/17; A61K 40/11; A61K 40/31; A61K 40/32; C07K 14/4702; C12N 5/0636; C12N 15/86; C12N 2510/00; C12N 2740/15043; C12N 2740/16043; C12N 2750/14321; C12N 2830/002; C12N 2830/42; C12N 2830/48; C12N 2830/60

(57) ABSTRACT

A polynucleotide regulatory sequence that can be used to increase the protein expression level of a protein expression system, a protein expression vector or a protein expression system comprising the regulatory sequence, as well as the use thereof are also provided. The use of the regulatory sequence can increase the expression level of a heterologous protein. When used in a viral expression system, the regulatory sequence can also increase the titer of the virus particles.

### CROSS REFERENCE TO RELATED APPLICATION

This application claims priority benefits of International Application No. PCT/CN2023/071666, filed on Jan. 10, 2023, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: IEC232082PCT. Sequence listing.xml, date recorded: Jan. 10, 2024, size: 23 KB).

### TECHNICAL FIELD

The present disclosure relates to the fields of molecular biology and bioengineering. In particular, the present disclosure relates to a regulatory sequence for expressing a gene of interest (GOI). The present disclosure also relates to an expression system comprising the regulatory sequence, as well as the use of the expression system.

### BACKGROUND

The main indicators for evaluating the virus (e.g., lentivirus) protein expression system include: virus titer, transduction rate (the positive rate after infection of target cells), protein expression levels, and purification process adaptability, etc. Among them, the virus titer indicates the virus particle's gene delivery ability, which affects the cost of cell therapy. Thus, it is desirable to increase the virus titer.

Strategies for increasing virus titer usually include the following aspects: 1) optimizing the culture system for the packaging cell; 2) improving the downstream processes including purification and freezing methods to reduce losses and improve recovery of virus particles; 3) concentrating the virus supernatant (e.g., by ultracentrifugation); 4) optimizing the vector sequences. After years of development, the culture system of packaging cells has reached a stable and efficient state, with limited room for improvement, and the titer in the cell culture supernatant is usually about 10⁷ Tu/ml. Similarly, after years of development, the downstream processes and methods for concentrating the virus supernatant have little room for improvement. Thus, the present disclosure focuses on the optimization of vector sequences.

With the continuous development in the field of cell therapy, the design of CAR (chimeric antigen receptor) molecules or some other therapeutic agents is becoming more and more refined, and the size and complexity of the target gene fragment continues to increase with the incorporation of cytokines/chemokines/co-expressed antibodies. Commonly used lentiviral vectors do not perform well for large or unique target gene virus titers will often drop significantly. Therefore, further optimization of the vector sequence is needed.

### SUMMARY

The present disclosure provides a regulatory nucleic acid sequence (e.g., DNA or RNA) that can be used to enhance the expression of the heterologous protein, wherein the regulatory sequence is capable of being used for enhancing expression level of the heterologous proteins of the expression systems.

In one aspect, the disclosure is related to a regulatory sequence, comprising: (a) an EF1α promoter; (b) a small intron region of the minute virus of mice (MVM); and (c) a NF-κB binding site.

In some embodiments, the EF1α promoter is a truncated EF1α promoter.

In some embodiments, the EF1α promoter does not contain any intron sequence.

In some embodiments, the EF1α promoter does not contain nucleotides 407-757 of the full length EF1α promoter.

In some embodiments, the EF1α promoter comprises a sequence that is at least 70%, 80%, 85%, 90% or 100% identical to SEQ ID NO: 2, 5 or 6.

In some embodiments, the small intron region of MVM comprises a sequence that is at least 70%, 80%, 85%, 90% or 100% identical to SEQ ID NO: 3 or 13.

In some embodiments, the NF-κB binding site comprises a NF-κB binding sequence that is at least 70%, 80%, 85%, 90% or 100% identical to SEQ ID NO: 8.

In some embodiments, the regulatory sequence comprises a sequence that is at least 70%, 80%, 85%, 90% or 100% identical to SEQ ID NO: 4 or 14.

In some embodiments, the regulatory sequence further comprises a WPRE.

In some embodiments, the regulatory sequence does not comprise a WPRE.

In some embodiments, the NF-κB binding site comprises at least three copies of a NF-κB binding sequence (e.g., SEQ ID NO: 8).

In some embodiments, the NF-κB binding site comprises about 4 to 6 copies of a NF-κB binding sequence (e.g., SEQ ID NO: 8).

In some embodiments, the NF-κB binding site comprises 5 copies of a NF-κB binding sequence (e.g., SEQ ID NO: 8), and the NF-κB binding site is at least 70%, 80%, 85%, 90% or 100% identical to SEQ ID NO: 1. In some embodiments, the NF-κB binding site further comprises a linker sequence (e.g., SEQ ID NO: 10). In some embodiments, the NF-κB binding site is at least 70%, 80%, 85%, 90% or 100% identical to SEQ ID NO: 11.

In some embodiments, the total length of the regulatory sequence is less than 1200 bp.

In some embodiments, the regulatory sequence comprises, from the 5′ end to the 3′ end: (a) the NF-κB binding site; (b) the EF1α promoter; and (c) the small intron region of MVM.

In some embodiments, the regulatory sequence comprises, from the 5′ end to the 3′ end: (a) the small intron region of MVM; (b) the NF-κB binding site; and (c) the EF1α promoter.

In some embodiments, the regulatory sequence comprises, from the 5′ end to the 3′ end: (a) the NF-κB binding site; (b) the small intron region of MVM; and (c) the EF1α promoter.

In one aspect, the disclosure is related to a vector, comprising: the regulatory sequence described herein and a gene of interest (GOI).

In some embodiments, the GOI encodes a chimeric antigen receptor (CAR), a modified T-cell receptor (TCR), an antibody, a fusion protein, an enzyme, a soluble protein, a membrane protein, a structural protein, a ribosome protein, a zymogen, a cell surface receptor protein, a transcriptional regulatory protein, a translational regulatory protein, a chromatin protein, a hormone, a cell cycle regulatory protein, a G protein, a neuroactive peptide, an immunomodulatory protein, a blood component protein, an ion gate protein, a heat shock protein, a dihydrofolate reductase, an antibiotic resistance protein, a functional fragment of any one of the proteins, and any combination thereof.

In some embodiments, the vector is a lentivirus vector.

In some embodiments, the vector further comprises a 5′ long terminal repeat (LTR) and/or a 3′ LTR.

In some embodiments, the vector further comprises a Psi (T) packaging signal.

In some embodiments, the vector further comprises a heterologous polyadenylation sequence.

In some embodiments, the vector further comprises a cytomegalovirus (CMV) promoter.

In some embodiments, the vector comprises, from the 5′ end to the 3′ end: (a) the CMV promoter, (b) the 5′ LTR, (c) the regulatory sequence, (d) the GOI, and (e) the 3′ LTR.

In some embodiments, the vector comprises, from the 5′ end to the 3′ end: (a) the CMV promoter, (b) the 5′ LTR, (c) the regulatory sequence, (d) the GOI, (e) the WPRE, and (f) the 3′ LTR.

In some embodiments, the vector further comprises a Rev-responsive element (RRE).

In some embodiments, the vector further comprises a central polypurine tract (cPPT).

In one aspect, the disclosure is related to a packaging cell comprising the vector described herein.

In one aspect, the disclosure is related to a virus particle produced by the packaging cell described herein.

In one aspect, the disclosure is related to a virus particle comprising a nucleic acid comprising the regulatory sequence described herein.

In some embodiments, the virus particle is a lentivirus particle.

In some embodiments, the nucleic acid is RNA.

In one aspect, the disclosure is related to a method of expressing a protein of interest, comprising:
(a) producing virus particles using the packaging cell described herein;
(b) infecting a host cell with the virus particles; and
(c) expressing a protein of interest using the host cell.

In some embodiments, the method can increase the expression level of the protein of interest by about or at least 10%, 20%, 30%, 40%, 50%, 60%, 70%, 80%, 90%, 100%, 120%, 140%, 160%, 180%, 200%, 300%, 400%, 500%, 600%, or 700%, as compared to a control sequence that is different from the TENM sequence.

In some embodiments, the method can increase the titer of the virus particles by about or at least 10%, 20%, 30%, 40%, 50%, 60%, 70%, 80%, 90%, 100%, 120%, 140%, 160%, 180%, 200%, 300%, 400%, 500%, 600%, or 700%, as compared to a control sequence that is different from the TENM sequence.

In one aspect, the disclosure is related to a method of making an immune cell, comprising (a) isolating immune cells from a subject; and (b) infecting the isolated immune cells using the virus particles described herein.

In one aspect, the disclosure is related to an immune cell comprising the regulatory sequence described herein.

In some embodiments, the immune cell is selected from the group consisting of a cytotoxic T cell, a helper T cell, a natural killer (NK) cell, an iNK-T cell, an NK-T like cell, an αβT cell and a γδT cell.

In some embodiments, the immune cell comprises a gene of interest that is operably linked to the regulatory sequence described herein, wherein the gene of interest encodes a chimeric antigen receptor (CAR) or a modified T-cell receptor (TCR).

In some embodiments, the immune cell expresses the chimeric antigen receptor (CAR) or modified T-cell receptor (TCR).

In one aspect, the disclosure is related to a method of treating a subject having cancer, the method comprising administering a therapeutically effective amount of a composition comprising the virus particle described herein, or the immune cell described herein, to the subject.

In some embodiments, the cancer is solid tumor.

In some embodiments, the cancer is hematologic cancer.

In one aspect, the disclosure is related to a pharmaceutical composition comprising the virus particle described herein or the immune cell described herein, and a pharmaceutically acceptable carrier.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 is a schematic diagram of relevant vector designs.

FIG. 2A illustrates the influence of EF1α promoter (EF1αΔ407-757 promoter and EF1αS promoter) on virus titer.

FIG. 2B illustrates the influence of EF1α promoter (EF1αΔ407-757 promoter and EF1αS promoter) on protein expression levels.

FIG. 3A illustrates the influence of MVM intron on virus titer.

FIG. 3B illustrates the influence of MVM intron on protein expression levels.

FIG. 4A illustrates the influence of NF-κB binding site on virus titer.

FIG. 4B illustrates the influence of NF-κB binding site on protein expression levels.

FIG. 4C illustrates the influence of NF-κB binding site with different copies on virus titer.

FIG. 4D illustrates the influence of NF-κB binding site with different copies on protein expression level.

FIG. 5A illustrates the influence of the order of the EF1α promoter, MVM intron, and NF-κB binding site on virus titer (CAR #1).

FIG. 5B illustrates the influence of the order of the EF1α promoter, MVM intron, and NF-κB binding site on protein expression levels (CAR #1).

FIG. 6A illustrates the influence of the TENM promoter on virus titer of supernatant (CAR #3).

FIG. 6B illustrates the influence of the TENM promoter on titer of concentrated virus (CAR #3).

FIG. 6C illustrates the influence of the TENM promoter on protein expression levels (CAR #3).

FIG. 7A illustrates the influence of the TENM promoter on virus titer of supernatant (CAR #7).

FIG. 7B illustrates the influence of the TENM promoter on titer of concentrated virus (CAR #7).

FIG. 7C illustrates the influence of the TENM promoter on protein expression levels (CAR #7).

FIG. 8A illustrates the influence of the TENM promoter on virus titer of supernatant (CAR #8).

FIG. 8B illustrates the influence of the TENM promoter on titer of concentrated virus (CAR #8).

FIG. 8C illustrates the influence of the TENM promoter on protein expression levels (CAR #8).

FIG. 9 illustrates the influence of the TENM promoter on titer of concentrated virus (CAR #2).

FIG. 10A-10B illustrate the influence of the TENM promoter on virus transduction efficiency (CAR #2).

FIG. 10C-10D illustrate the influence of the TENM promoter on protein expression level (CAR #2).

FIG. 11A-11B illustrate the influence of the TENM promoter on in vitro tumor killing (CAR #2).

FIG. 11C illustrates the influence of the TENM promoter on T cell phenotype (CAR #2).

FIG. 12 illustrates the influence of the TENM promoter on in vivo tumor killing (CAR #2).

FIG. 13 illustrates the influence of the TENM promoter on transcription level (CAR #2).

FIG. 14A illustrates the influence of the TENM promoter on T cell diameter post IL-2 deprivation (CAR #6).

FIG. 14B illustrates the influence of the TENM promoter on T cell expansion post IL-2 deprivation (CAR #6).

FIG. 14C illustrates the influence of the TENM promoter on T cell viability post IL-2 deprivation (CAR #6).

FIG. 14D illustrates the influence of the TENM promoter on T cell phenotype post IL-2 deprivation (CAR #6).

FIG. 15A is a schematic diagram of nest PCR study.

FIG. 15B illustrates the influence of the TENM promoter on integrity of virus insertion on chromosome.

FIG. 15C illustrates the influence of the TENM promoter on accuracy of virus integration on chromosome.

FIG. 16A illustrates the influence of hEF1α promoter on frequency of integration loci.

FIG. 16B and FIG. 16C illustrate the influence of the TENM promoter on frequency of integration loci.

FIG. 17A illustrates the influence of hEF1α promoter on distribution of integration loci on chromosome.

FIG. 17B and FIG. 17C illustrate the influence of the TENM promoter on distribution of integration loci on chromosome.

FIG. 18A illustrates the influence of hEF1α promoter on integration frequency in oncogenes.

FIG. 18B and FIG. 18C illustrate the influence of the TENM promoter on integration frequency in oncogenes.

FIG. 19 is a schematic diagram of relevant vector designs (MVM intron variants).

FIG. 20A illustrates the influence of the TENM promoter on virus titer (CAR #3).

FIG. 20B illustrates the influence of the TENM promoter on protein expression level (CAR #3).

FIG. 21A illustrates the influence of the TENM promoter on virus titer (CAR #4).

FIG. 21B illustrates the influence of the TENM promoter on protein expression level (CAR #4).

FIG. 22 illustrates the influence of the TENM promoter on virus titer (CAR #5).

FIG. 23A illustrates the influence of the TENM promoter on virus titer (CAR #6).

FIG. 23B illustrates the influence of the TENM promoter on protein expression level (CAR #6).

FIG. 24A illustrates the influence of the TENM promoter on transduction efficiency (CAR #6).

FIG. 24B illustrates the influence of the TENM promoter on protein expression level (CAR #6).

FIG. 25 illustrates the influence of the TENM promoter on virus transduction efficiency (CAR #6).

FIG. 26 is a schematic diagram of relevant vector designs.

FIG. 27A-27C illustrate the influence of the TENM promoter on protein transcription termination.

FIG. 28 shows relevant nucleotide sequences.

### DETAILED DESCRIPTION

Lentivirus and AAV (adeno-associated virus) expression cassettes, prominently used in gene therapy, can be designed for target specificity and transgene expression levels. For example, cis-acting elements such as introns, WPRE, polyadenylation signals, and the CMV enhancer can be modified to enhance transgene expression. Expression cassettes require thoughtful design due to foreign DNA packaging size constraints of AAV and lentivirus, approximately 4.1-4.9 kb and 8-9 kb, respectively. However, many regulatory sequences that can effectively induce transcription and translation are quite long. Given the size constraint, it can be advantageous to reduce the length of the regulatory sequences. Different cis-acting elements have been engineered into expression cassettes to enhance transgene expression.

The present disclosure provides a polynucleotide regulatory sequence that can be used to increase the transcription level of the sequence of interest and/or the protein expression level. The use of the regulatory sequence can increase the expression level of a heterologous protein in a host cell greatly. When used in a viral expression system, the regulatory sequence can also increase the production of virus particles, e.g., the titer of the virus from the packaging cells. Particularly, the results of the examples show that when the target gene sequence is large, using the regulatory sequence as described herein can significantly increase the virus titer while keeping a high expression level. The regulatory sequence can not only be applied to cell therapy and gene therapy products, but also can be used to express target protein/antibody/vaccine antigen in the field of scientific research and industrial production, and has high clinical and commercial application value.

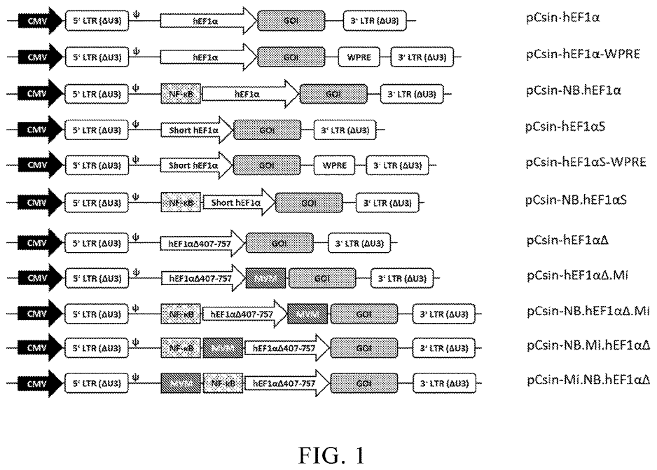

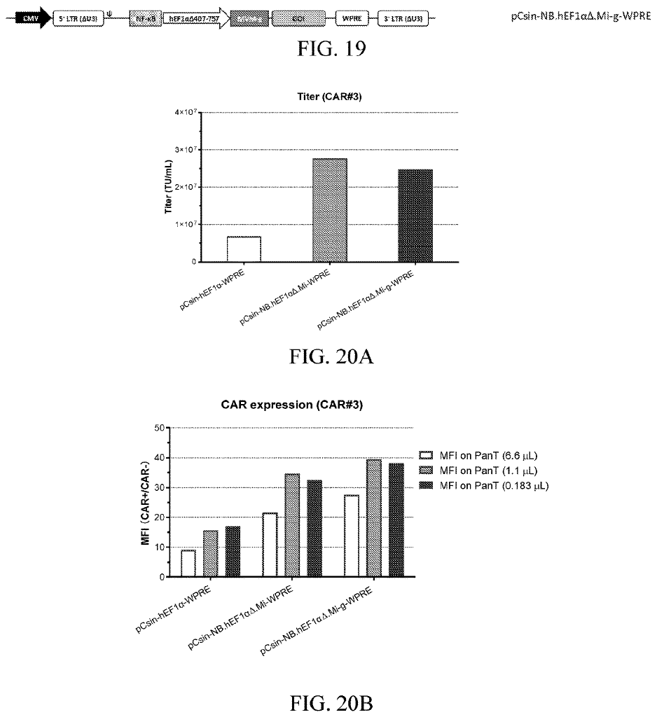

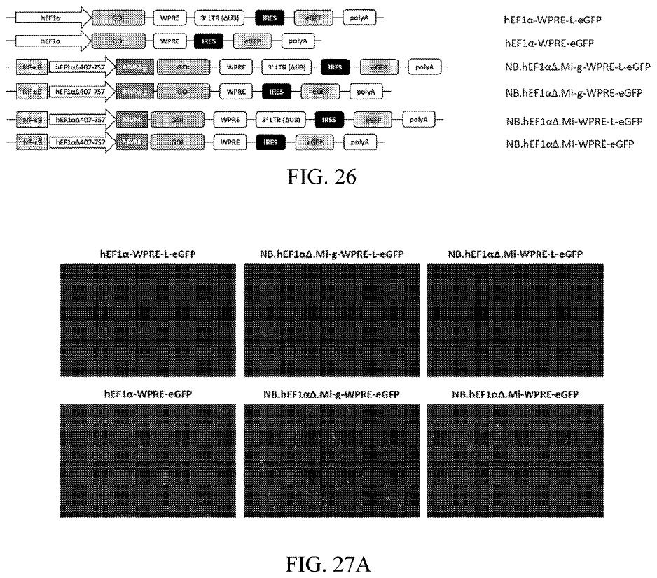

A typical third-generation lentiviral packaging vector architecture is shown in FIG. 1, FIG. 19 and FIG. 26. This kind of vector can still be used when packaging shorter target gene fragments (e.g., fluorescent protein reporter genes), but the virus titer is not high for large or complex proteins (e.g., CAR). To address this problem, the external promoter located in front of the 5′ LTR (Long Terminal Repeat) can be replaced by the CMV promoter (cytomegalovirus promoter) to improve the virus titer. Further, the WPRE (Woodchuck Hepatitis Virus Posttranscriptional Regulatory Element) is removed to improve the virus titer. However, for larger target gene fragments, the virus titer is still low, and the removing of the WPRE may also lead to decreased expression level of the target gene after transduction of cells.

Human EF1α promoter (human elongation factor-1 alpha promoter) is a strong promoter with a high expression level, and can work stably in vivo and in vitro. Compared with other promoters, the EF1α promoter is not easy to be silenced and shows good safety performance, because of its non-viral origin. In actual use, the EF1α promoter selection is usually the full-length type (SEQ ID NO: 5) or only the core promoter sequence (SEQ ID NO: 6). When the target sequence is large or complex, neither version of the EF1α promoter can balance the virus titer and protein expression level. While the full-length EF1α promoter has better protein expression levels, but the virus titer is low. The core promoter sequence, even when combined with WPRE, leads to decreased protein expression levels comparing to the full-length EF1α promoter. Considering that the internal regulatory sequence between the LTR sequences is the part that integrates into the host cell genome, optimization of the internal regulatory sequence is desirable.

### Regulatory Sequences

The term “regulatory sequence” in the present disclosure refers to a polynucleotide sequence involved in the regulation of gene transcription and/or expression. The polynucleotide can include a promoter, an enhancer, an insulator, and the like. In some embodiments, the regulatory sequence comprises or consists of any one or the combination of the following: (a) a promoter (e.g., an EF1α promoter); (b) a small intron region of the minute virus of mice (MVM); and (c) a NF-κB binding site.

In some embodiments, the regulatory sequence comprises or consists of (a) an EF1α promoter; and (b) a small intron region of the minute virus of mice (MVM). In some embodiments, the regulatory sequence comprises or consists of (a) an EF1α promoter; and (b) a NF-κB binding site. In some embodiments, the regulatory sequence comprises or consists of (a) a small intron region of the minute virus of mice (MVM); and (b) a NF-κB binding site.

In some embodiments, the regulatory sequence is a truncated hEF1α promoter armored with NF-κB binding site and MVM small intron (TENM), which includes (1) an EF1α promoter, (2) a MVM intron, and (3) NF-κB binding site.

It is believed that the present disclosure, for the first time, introduces the NF-κB binding site in the lentiviral packaging process to effectively maintain or increase the expression level of the target gene. The present disclosure showed that its effect is better than or equal to that of the EF1α promoter and WPRE element. Deleting the WPRE element at the same time can further improve the safety of the vector and virus in clinical use.

It is also believed that the present disclosure, for the first time, introduces the MVM intron in the lentivirus packaging process to increase the titer of the lentivirus. Combined with the above-mentioned EF1αΔ407-757 promoter, the introduction of MVM intron creates a regulatory sequence with double introns. The MVM intron has been used in clinical trials, and no safety issues have been reported, so it has good clinical safety and use value.

The regulatory sequence described herein can efficiently increase the titer of lentivirus particles while maintaining or increasing the expression intensity of the target gene. In some embodiments, the length of the regulatory sequence containing 4~6 copies (e.g., 5 copies) of NF-κB binding sequence is 1,153 bp, which is significantly shorter than the EF1α promoter and WPRE element (totally 1,927 bp). Thus, the regulatory sequence described herein not only provides better performance in terms of titer, but also can provide a larger target gene packaging space for the lentivirus. In some embodiments, the regulatory sequence can be used in a virus vector (e.g., lentivirus vector). In some embodiments, the regulatory sequence can be used in various cells, including but not limited to, HEK293T cells, CHO cells and Vero cells. In some embodiments, the regulatory sequence can be used in various gene editing technologies, including but not limited to CRISPR, TALEN, Piggybac, sleeping beauty, retroviruses, adenoviruses, and adeno-associated viruses.

Further, as the regulatory sequence described herein does not contain any element with significant risks, the regulatory sequence can be used in vectors to treat diseases (e.g., cell therapy or gene therapy) or to express therapeutic proteins (e.g., antibodies). In some embodiments, the regulatory sequence can be used in a cell therapy (e.g., CAR-T, CAR-NK, or TCR-T). In some embodiments, the regulatory sequence can be used in a gene therapy. In some embodiments, the regulatory sequence can be used to express an antibody. In some embodiments, the regulatory sequence can be used to express a vaccine.

### 1. Promoter (e.g., EF1α Promoter)

Promoters are regions of DNA that leads to initiation of transcription of a polynucleotide encoding a polypeptide. Promoters are located near the transcription start sites of the coding sequence, upstream on the DNA (towards the 5′ region of the sense strand of the coding sequence). Promoters are a major cis-acting element within the vector genome design that can dictate the overall strength of expression as well as cell-specificity. Attaching RNA polymerase to the binding site of the promoter activates the transcription of the desired gene. Constitutive promoters such as the human elongation factor 1α-subunit (EF1α), immediate-early cytomegalovirus (CMV), chicken 3-actin (CBA) and its derivative CAG, the f glucuronidase (GUSB), or ubiquitin C (UBC) can be used to promote expression in most tissues. Generally, CBA and CAG promote the larger expression among the constitutive promoters; however, their size of ~1.7 kb in comparison to CMV (~0.8 kb) or EF1α (~1.2 kb) limits its use in vectors with packaging constraints. The GUSB or UBC promoters can provide ubiquitous gene expression with a smaller size of 378 bp and 403 bp, respectively, but they are considerably weaker than the CMV or CBA promoter. Selecting the proper promoter is crucial to obtaining effective transgene production within the cells.

A typical mammalian promoter may have a 50-100 base pair core region to which the basic transcription machinery binds, and an enhancer region to which one or more transcriptional activator proteins (transactivators) may bind. The number and type of transactivators that are able to bind at an enhancer depends on which specific binding sites are present. Core promoters are made up of various different elements, of which there are two categories: canonical and non-canonical. Canonical core promoter elements include: TATA box, the initiator (Inr), the TFIIB recognition element (BRE), downstream promoter element (DPE) and downstream core element (DCE). These elements can be found within the core promoters of many but not all protein-coding genes. The TATA box (sequence TATAA) is usually found 20-30 bp upstream of the transcription start site (TSS) and acts as a binding site for the TFIID general transcription factor.

Non-canonical core promoter elements include the CpG island, the ATG desert and the transcription initiation platform (TIP). CpG islands generally span a 500-2,000 bp stretch of DNA that contains a relatively high proportion of CpG dinucleotides. CpG dinucleotides would normally be methylated on the C residue, reducing transcription, but within CpG islands they remain unmethylated, promoting transcription. An ATG desert is a region of DNA with a lower frequency of ATG trinucleotides than surrounding regions. They extend approximately 1000 bp up and downstream of the TSS and are generally associated with promoters that do not contain TATA boxes.

The present disclosure provides a nucleic acid comprising a promoter, optionally with 1, 2, 3, 4, 5, or more than 5 elements as described herein. In some embodiments, the promotor is a SV40 promoter, CMV promoter (e.g., human CMV promoter, mouse CMV promoter), retinoschisin promoter, a rhodopsin promoter, a rhodopsin kinase promoter, a CRX promoter, an interphotoreceptor retinoid binding protein (IRBP) promoter, a human elongation factor 1α-subunit (EF1α), or variants thereof (e.g., a truncated EF1α). Any promoter that allows tissue-specific expression of an encoded protein can also be used. A strong promoter is one that initiates transcription with a high frequency and can be a very useful tool.

In some embodiments, the promoter is an EF1α promoter. As used herein, the EF1α promoter refers to the promoter for the EF1α gene. The term includes both the wildtype promoter and the variants thereof, wherein the variants retain the promoter activity. Human EF1α promoter (human elongation factor-1 alpha promoter) is a strong promoter with a high expression level, and can work stably in vivo and in vitro. The full length of the human EF1α promoter sequence is shown in SEQ ID NO: 5, and the length of the core promoter is about 230 bp (SEQ ID NO: 6). The intron sequence of the EF1α promoter plays an important role in the regulation of the promoter and the process of mRNA splicing. Typically, the EF1α promoter selected is the full-length EF1α promoter (SEQ ID NO: 5) or only the core promoter sequence (SEQ ID NO: 6). When the target sequence is large or complex, neither the full length EF1α promoter or the core promoter sequence can balance the titer and the expression level well: the full-length EF1α promoter creates better expression levels, but the titer is lower; and the truncated version (even with the WPRE element) creates significantly decreased expression levels. Thus, further modifications of the EF1α promoter are needed. The present disclosure provides variants of the EF1α promoter. In some embodiments, the regulatory sequence contains a truncated EF1α promoter, EF1αΔ407-757 (SEQ ID NO: 2).

In some embodiments, the EF1α promoter is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2, SEQ ID NO: 5, or SEQ ID NO: 6.

In some embodiments, the EF1α promoter is less than 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1,000 bp, 1,050 bp, 1,100 bp, 1,150 bp, 1,200 bp, 1,250 bp, 1,300 bp, or 1,350 bp.

In some emboldens, the EF1α promoter is at least 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1,000 bp, 1,050 bp, 1,100 bp, 1,150 bp, 1,200 bp, 1,250 bp, 1,300 bp, or 1,350 bp.

In some embodiments, the EF1α promoter is 200~400 bp, 400~600 bp, 600~800 bp, 800~1,000 bp, 1,000~1,200 bp, 1200~1400 bp, 1,400~1,600 bp, 200~500 bp, 500~1,000 bp, 800~1,200 bp, 700~1,300 bp, or 1,000~1,500 bp.

In some embodiments, the EF1α promoter has at least 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% of the length of the full length of EF1α promoter.

In some embodiments, the EF1α promoter has no more than 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% of the length of the full length of EF1α promoter.

In some embodiments, the promoter is located upstream (at the 5′ end side) the gene of interest.

In some embodiments, the regulatory sequence comprises or consists of the EF1α promoter and one or more additional elements as described herein, e.g., introns, and/or NF-κB binding site.

### 2. Intron (e.g., MVM Intron)

The presence of an intron or intervening sequence in mRNA is important for mRNA processing and increased transgene expression. For example, the presence of the SV40 intron between the promoter and the transgene, in an AAV expression cassette, gives a 2-fold increase of transgene expression under the CMV promoter and enhancer in lung carcinoma cells. A variety of introns between the promoter and transgene can be used to enhance transgene expression in mice using AAV2. Although intron presence in eukaryotic genes provides clear benefit in expression efficiency over intron-less genes, most introns are much larger than the coding exons so identifying an intronic element small enough to fit within the transgene construct without preventing its packaging within the AAV virus is a challenge. In some embodiments, the MVM (minute virus of mice) intron can increase transgene expression more than 80-fold (over no intron). The MVM intron is extremely small and thus called MVM small intron. A small intron region from MVM of 92 nucleotides in length has shown the most robust potentiation of gene expression from a panel of several small intron sequences.

As used herein, the terms “MVM intron”, “MVM small intron” or “a small intron region of the MVM” are used interchangeably herein to refer to the intron element to include in an expression cassette to increase transgene expression.

In some embodiments, the regulatory sequence contains a MVM intron (SEQ ID NO: 3 or SEQ ID NO: 13), which is a small intron region of the MVM. The present disclosure provides a nucleic acid comprising an intron (e.g., MVM intron), optionally with 1, 2, 3, 4, 5, or more than 5 elements as described herein. As used herein, the MVM intron includes the wildtype MVM intron and the variants thereof, wherein the variants retain the activity of MVM intron sequence (e.g., increasing transcription level).

In some embodiments, the MVM intron sequence is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3.

In some embodiments, the MVM intron sequence is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 13.

In some embodiments, the intron (e.g., MVM intron) is less than 25 bp, 30 bp, 35 bp, 400 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 130 bp, or 150 bp.

In some embodiments, the intron (e.g., MVM intron) is at least 25 bp, 30 bp, 35 bp, 400 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 130 bp, or 150 bp.

In some embodiments, the intron is located upstream (at the 5′ end side) the gene of interest.

In some embodiments, the intron is located downstream (e.g., at the 3′ end side) of the promoter.

In some embodiments, the regulatory sequence comprises or consists of the intron (e.g., MVM intron) and one or more additional elements as described herein, e.g., the promoter and/or NF-κB binding site.

### 3. NF-κB Binding Site

Nuclear factor kappa-light-chain-enhancer of activated B cells (NF-κB) is a protein complex that controls transcription of DNA, cytokine production and cell survival. Upon activation, the NF-κB complex enters the nucleus where it can enhance the expression of specific genes that have DNA-binding sites for NF-κB (NF-κB binding site) nearby. For example, NF-κB has been demonstrated to mediate rat intrarenal angiotensinogen (AGT) transcription. An acute-phase response element was identified in the rat AGT promoter region, which consists of a composite NF-κB and CCAAT/enhancer protein binding site.

In some embodiments, the regulatory sequence includes a NF-κB binding site. In some embodiments, the NF-κB binding site comprises one, two or more copies of a NF-κB binding sequence (e.g., SEQ ID NO: 8). In some embodiments, the NF-κB binding site comprises five copies of a NF-κB binding sequence, forming a 5×NF-κB binding site (e.g., SEQ ID NO: 1). In some embodiments, the NF-κB binding site further comprises a linker sequence (e.g., SEQ ID NO: 10). In some embodiments, the NF-κB binding site is at least 70%, 80%, 85%, 90% or 100% identical to SEQ ID NO: 11. By repetition of the NF-κB binding sequence, an increase of the activity is possible.

In some embodiments, the NF-κB binding site comprises 1, 2, 3, 4, 5, 6, 7, 8, 9, 10, or more than 10 copies of a NF-κB binding sequence (e.g., SEQ ID NO: 8). As used herein, NF-κB binding sequence includes the wildtype NF-κB binding sequence (e.g., SEQ ID NO: 8) and the variants thereof, wherein the variants retain the activity of a NF-κB binding sequence (e.g., binding to NF-κB and/or increasing transcription level).

In some embodiments, the sequence of the NF-κB binding site is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1, SEQ ID NO: 8, or SEQ ID NO: 11.

In some embodiments, the length of the NF-κB binding site is less than or equal to 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 130 bp, or 150 bp.

In some embodiments, the length of the NF-κB binding site is at least 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 130 bp, or 150 bp.

In some embodiments, the NF-κB binding site is located upstream (at the 5′ end side) the gene of interest.

In some embodiments, the regulatory sequence comprises or consists of the NF-κB binding site and one or more additional elements as described herein, e.g., the promoter and/or MVM intron.

### 4. Post-Transcriptional Regulatory Elements (e.g., WPRE)

Viral post-transcriptional regulatory elements (PREs) are important for viral gene expression; these cis-acting elements are required for nuclear export of intronless viral RNA. Both HPRE (Hepatitis B Virus PRE, 533 bp) and WPRE (Woodchuck Hepatitis Virus PRE, 600 bp) can increase the level of transgene expression by 6.1-fold and 8.6-fold, respectively. The difference in expression was determined to be due to an additional sequence element in WPRE. WPRE can be shortened (to 247 bp), as demonstrated in neurons in vivo and in vitro, and it still offers sufficient transgene expression. In cultured human cells using lentiviral and AAV vectors, WPRE can increase CMV promoter driven transgene expression up to 8-fold. In vivo studies have also shown an increase of PPE, PDGF, NSE, or CMV promoter-driven transgene expression by the presence of WPRE.

The present disclosure provides a nucleic acid comprising a WPRE, optionally with 1, 2, 3, 4, 5, or more than 5 elements as described herein. As used herein, the WPRE includes the wildtype WPRE and the variants thereof, wherein the variants retain the activity of the WPRE (e.g., increasing protein expression level). In some embodiments, the WPRE is the wildtype WPRE (e.g., SEQ ID NO: 9). In some embodiments, the WPRE is a mutant WPRE (e.g., SEQ ID NO: 12).

In some embodiments, the WPRE is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 9. In some embodiments, the WPRE is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12.

In some embodiments, the WPRE is less than or equal to 400 bp, 450 bp, 500 bp, 550 bp, 591 bp, 600 bp, 650 bp, 700 bp, 750 bp, 800 bp, 850 bp, or 900 bp.

In some embodiments, the WPRE is at least 400 bp, 450 bp, 500 bp, 550 bp, 600 bp, 650 bp, 700 bp, 750 bp, 800 bp, 850 bp, or 900 bp.

In some embodiments, the regulatory sequence comprises or consists of the WPRE and one or more additional elements as described herein, e.g., the promoter and/or NF-κB binding site.

In some embodiments, the WPRE is located downstream (at the 3′ end side) the gene of interest.

In some embodiments, the regulatory sequence does not have the WPRE.

5. TENM (Truncated hEF1α Promoter Armored with NF-κB Binding Site and MVM Small Intron)

As used in the present disclosure, “TENM” or “truncated hEF1α promoter armored with NF-κB binding site and MVM small intron” refers to a regulatory sequence that comprises (1) a truncated hEF1α promoter, (2) a NF-κB binding site and (3) a MVM intron. In some embodiments, exemplary TENM structures can be found in FIG. 1, FIG. 19 and FIG. 26.

In some embodiments, said TENM sequence has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 4. In some embodiments, the TENM sequence has at least or about 1, 2, 3, 4, 5, 6, 7, 8, 9, or 10 conservative mutation relative to a sequence set forth in SEQ ID NO: 4. In some embodiments, the TENM sequence differs from the sequence set forth in SEQ ID NO: 4 by at least or about 1, 2, 3, 4, 5, 6, 7, 8, 9, or 10 nucleotides.

In some embodiments, said TENM sequence has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 14. In some embodiments, the TENM sequence has at least or about 1, 2, 3, 4, 5, 6, 7, 8, 9, or 10 conservative mutation relative to a sequence set forth in SEQ ID NO: 14. In some embodiments, the TENM sequence differs from the sequence set forth in SEQ ID NO: 14 by at least or about 1, 2, 3, 4, 5, 6, 7, 8, 9, or 10 nucleotides.

In some embodiments, the truncated hEF1α promoter has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence of any of SEQ ID NO: 2, 5, or 6. In some embodiments, the truncated hEF1α promoter has at least or about 1, 2, 3, 4, 5, 6, 7, 8, 9, or 10 conservative mutation relative to a sequence of any of SEQ ID NO: 2, 5, or 6. In some embodiments, the truncated hEF1α promoter differs from any of SEQ ID NOs: 2, 5, or 6 by at least or about 1, 2, 3, 4, 5, 6, 7, 8, 9, or 10 nucleotides.

In some embodiments, said MVM intron has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 3. In some embodiments, the MVM intron has at least or about 1, 2, 3, 4, 5, 6, 7, 8, 9, or 10 conservative mutation relative to a sequence set forth in SEQ ID NO: 3. In some embodiments, the MVM intron differs from the sequence set forth in SEQ ID NO: 3 by at least or about 1, 2, 3, 4, 5, 6, 7, 8, 9, or 10 nucleotides.

In some embodiments, said MVM intron has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 13. In some embodiments, the MVM intron has at least or about 1, 2, 3, 4, 5, 6, 7, 8, 9, or 10 conservative mutation relative to a sequence set forth in SEQ ID NO: 13. In some embodiments, the MVM intron differs from the sequence set forth in SEQ ID NO: 13 by at least or about 1, 2, 3, 4, 5, 6, 7, 8, 9, or 10 nucleotides.

In some embodiments, said NF-κB binding site has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 1. In some embodiments, the NF-κB binding site has at least or about 1, 2, 3, 4, 5, 6, 7, 8, 9, or 10 conservative mutation relative to a sequence set forth in SEQ ID NO: 1. In some embodiments, the NF-κB binding site differs from the sequence set forth in SEQ ID NO: 1 by at least or about 1, 2, 3, 4, 5, 6, 7, 8, 9, or 10 nucleotides.

In some embodiments, said NF-κB binding site has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 11. In some embodiments, the NF-κB binding site has at least or about 1, 2, 3, 4, 5, 6, 7, 8, 9, or 10 conservative mutation relative to a sequence set forth in SEQ ID NO: 11. In some embodiments, the NF-κB binding site differs from the sequence set forth in SEQ ID NO: 11 by at least or about 1, 2, 3, 4, 5, 6, 7, 8, 9, or 10 nucleotides.

In some embodiments, said NF-κB binding site has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 8. In some embodiments, the NF-κB binding site has at least or about 1, 2, 3, 4, 5, 6, 7, 8, 9, or 10 conservative mutation relative to a sequence set forth in SEQ ID NO: 8. In some embodiments, the NF-κB binding site differs from the sequence set forth in SEQ ID NO: 8 by at least or about 1, 2, 3, 4, 5, 6, 7, 8, 9, or 10 nucleotides.

In some embodiments, variants can be made to any of the above-described sequences. In some embodiments, the TENM sequence or the variant thereof comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13 and/or SEQ ID NO:14.

In some embodiments, the TENM sequence can increase the expression amount of a heterologous protein by about or at least 10%, 20%, 30%, 40%, 50%, 60%, 70%, 80%, 100%, 120%, 140%, 160%, 180%, 200%, 300%, 400%, 500%, 600%, or 700% (e.g., as compared to a control sequence that is different from the TENM sequence).

In some embodiments, said regulatory sequence may also be selected from a nucleotide sequence comprising a TENM sequence and other sequences that may increase the level of protein expression.

In some embodiments, the TENM sequence and the polynucleotide encoding a polypeptide are operably linked together. In some embodiments, the TENM sequence, the polynucleotide encoding a polypeptide, and one or more additional regulatory elements are operably linked together. In some embodiments, the one additional regulatory element is a WPRE. The TENM sequence, the polynucleotide encoding a polypeptide, and the one or more additional regulatory elements that are operably linked together can have various orders. For example, the TENM sequence can be located before (e.g., from 5′ to 3′ on the sense strand of the coding sequence) or after the polynucleotide encoding a polypeptide (e.g., from 5′ to 3′ on the sense strand of the coding sequence). In some embodiments, there are at least or about 0, 1, 2, 3, 4, 5, 6, 7, 8, 9, 10, 100, 200, 300, 400, 500, 600, 700, 800, 900, 1K, 2K, 3K, 4K or 5K nucleotides between the TENM sequence and the polynucleotide encoding a polypeptide. In some embodiments, there are no more than 1, 2, 3, 4, 5, 6, 7, 8, 9, 10, 100, 200, 300, 400, 500, 600, 700, 800, 900, 1K, 2K, 3K, 4K or 5K nucleotides between the TENM sequence and the polynucleotide encoding a polypeptide. In some embodiments, the one or more additional regulatory elements are located between the TENM sequence and the sequence encoding a polypeptide.

In some embodiments, the regulatory sequence comprises or consists of a truncated hEF1α promoter (e.g., SEQ ID NO: 2), NF-κB binding site (e.g., SEQ ID NO: 1) and a MVM intron (e.g., SEQ ID NO: 3 or SEQ ID NO: 13). In some embodiments, the regulatory sequence comprises or consists of, from the 5′ end to the 3′ end, NF-κB binding site (e.g., SEQ ID NO: 1), a truncated hEF1α promoter (e.g., SEQ ID NO: 2), and a MVM intron (e.g., SEQ ID NO: 3 or SEQ ID NO: 13).

In some embodiments, the regulatory sequence comprises or consists of a truncated hEF1α promoter (e.g., SEQ ID NO: 2), NF-κB binding site (e.g., SEQ ID NO: 1) and a MVM intron (e.g., SEQ ID NO: 3 or SEQ ID NO: 13). In some embodiments, the regulatory sequence comprises or consists of, from the 5′ end to the 3′ end, NF-κB binding site (e.g., SEQ ID NO: 1), a MVM intron (e.g., SEQ ID NO: 3 or SEQ ID NO: 13), and a truncated hEF1α promoter (e.g., SEQ ID NO: 2).

In some embodiments, the regulatory sequence comprises or consists of a truncated hEF1α promoter (e.g., SEQ ID NO: 2), NF-κB binding site (e.g., SEQ ID NO: 1) and a MVM intron (e.g., SEQ ID NO: 3 or SEQ ID NO: 13). In some embodiments, the regulatory sequence comprises or consists of, from the 5′ end to the 3′ end, a MVM intron (e.g., SEQ ID NO: 3 or SEQ ID NO: 13), NF-κB binding site (e.g., SEQ ID NO: 1), and a truncated hEF1α promoter (e.g., SEQ ID NO: 2).

In some embodiments, the TENM sequence is less than or equal to 400 bp, 450 bp, 500 bp, 550 bp, 600 bp, 650 bp, 700 bp, 750 bp, 800 bp, 850 bp, 900 bp, 950 bp, 1,000 bp, 1,050 bp, 1,100 bp, 1,150 bp, 1,153 bp, 1,167 bp, 1,200 bp, 1,250 bp, 1,300 bp, 1,350 bp, 1,400 bp, 1,450 bp, 1,500 bp, 1,550 bp, 1,600 bp, 1,650 bp, 1,700 bp, 1,750 bp, 1,800 bp, 1,850 bp, 1,900 bp, 1,950 bp, or 2,000 bp.

In some embodiments, the TENM sequence is at least 400 bp, 450 bp, 500 bp, 550 bp, 600 bp, 650 bp, 700 bp, 750 bp, 800 bp, 850 bp, 900 bp, 950 bp, 1,000 bp, 1,050 bp, 1,100 bp, 1,150 bp, 1,153 bp, 1,167 bp, 1,200 bp, 1,250 bp, 1,300 bp, 1,350 bp, 1,400 bp, 1,450 bp, 1,500 bp, 1,550 bp, 1,600 bp, 1,650 bp, 1,700 bp, 1,750 bp, 1,800 bp, 1,850 bp, 1,900 bp, 1,950 bp, or 2,000 bp.

In some embodiments, the TENM sequence is located upstream (at the 5′ end side) of the gene of interest.

### 6. Other Regulatory Sequences

In some embodiments, the regulatory sequence described herein comprises a subset of (1) a truncated hEF1α promoter, (2) a NF-κB binding site and (3) a MVM intron. In some embodiments, the regulatory sequence described herein comprises a truncated hEF1α promoter and a MVM intron. In some embodiments, the regulatory sequence described herein comprises a subset of a NF-κB binding site and a MVM intron. In some embodiments, the regulatory sequence described herein comprises a truncated hEF1α promoter and a NF-κB binding site. In some embodiments, the regulatory sequence described herein comprises a hEF1α promoter and a MVM intron.

In some embodiments, the regulatory sequence described herein does not comprise a truncated hEF1α promoter. In some embodiments, the regulatory sequence described herein does not comprise a NF-κB binding site. In some embodiments, the regulatory sequence described herein does not comprise a MVM intron.

In some embodiments, the regulatory sequence comprises or consists of a full length hEF1α promoter (e.g., SEQ ID NO: 5).

In some embodiments, the regulatory sequence comprises or consists of a full length hEF1α promoter (e.g., SEQ ID NO: 5) and a WPRE (e.g., SEQ ID NO: 9 or SEQ ID NO: 12).

In some embodiments, the regulatory sequence comprises or consists of a full length hEF1α promoter (e.g., SEQ ID NO: 5) and a NF-κB binding site (e.g., SEQ ID NO: 1). In some embodiments, the regulatory sequence comprises or consists of, from the 5′ end to the 3′ end, a NF-κB binding site (e.g., SEQ ID NO: 1) and a full length hEF1α promoter (e.g., SEQ ID NO: 5).

In some embodiments, the regulatory sequence comprises or consists of a truncated hEF1α promoter (e.g., SEQ ID NO: 2).

In some embodiments, the regulatory sequence comprises or consists of a truncated hEF1α promoter (e.g., SEQ ID NO: 2) and a WPRE (e.g., SEQ ID NO: 9 or SEQ ID NO: 12).

In some embodiments, the regulatory sequence comprises or consists of a truncated hEF1α promoter (e.g., SEQ ID NO: 2) and a NF-κB binding site (e.g., SEQ ID NO: 1). In some embodiments, the regulatory sequence comprises or consists of, from the 5′ end to the 3′ end, a NF-κB binding site (e.g., SEQ ID NO: 1) and a truncated hEF1α promoter (e.g., SEQ ID NO: 2).

In some embodiments, the regulatory sequence comprises or consists of a truncated hEF1α promoter (e.g., SEQ ID NO: 2).

In some embodiments, the regulatory sequence comprises or consists of a truncated hEF1α promoter (e.g., SEQ ID NO: 2) and a MVM intron (e.g., SEQ ID NO: 3 or SEQ ID NO: 13). In some embodiments, the regulatory sequence comprises or consists of, from the 5′ end to the 3′ end, a truncated hEF1α promoter (e.g., SEQ ID NO: 2) and a MVM intron (e.g., SEQ ID NO: 3 or SEQ ID NO: 13).

### Activities of the Regulatory Sequences

The level of expression which has been promoted by the regulatory sequence described herein may be assayed by any suitable method. For example, for such an assay, the heterologous polynucleotide may be a selection marker gene (e.g. a neomycin resistance gene or hygromycin B phosphotransferase gene) or an expression reporter gene (e.g. LacZ, GFP (Green Fluorescence Protein), luciferase genes, etc.). In some embodiments, the heterologous polynucleotide is a chimeric antigen receptor (e.g., CAR #1, SEQ ID NO: 7).

In some embodiments, said heterologous polynucleotide has a sequence identity of at least or about 80%, 81%, 82%, 83%, 84%, 85%, 86%, 87%, 88%, 89%, 90%, 91%, 92%, 93%, 94%, 95%, 96%, 97%, 98%, 99%, or 100% (including all the ranges and percentages between these values) with the sequence set forth in SEQ ID NO: 7.

In some embodiments, the activity of the regulatory sequence described herein may be measured by operably inserting a reporter gene, such as the FLuc gene, downstream of the regulatory sequence described herein. The level of expression of firefly luciferase protein can then be used to indicate the level of expression. In some embodiments, the activity of the regulatory sequence described herein may be measured by operably inserting a gene of interest, such as CAR #1 (SEQ ID NO: 7), downstream of the regulatory sequence described herein. The level of expression of CAR #1 can then be used to indicate the level of expression.

The regulatory sequence described herein is capable of promoting expression of an operably-linked heterologous polynucleotide in a mammalian cell. Mammalian cells include mouse, rat, hamster, monkey and human cells. Examples of such cells include HEK cells and derivatives (e.g. HEK293, HEK293T, HEK293A), PerC6, 911, CHO, HCT116, HeLa, COS and VERO cells; cancer cells such as HepG2, A549, and MCF7; primary cells isolated from human or animal biopsies; immune cells (e.g., T cells and NK cells), and stem cells (including pluripotent cells such as embryonic stem cells and induced pluripotent stem (iPS) cells, as well as multipotent stem cells such as hematopoietic stem cells, mesenchymal stem cells, etc.).

In some embodiments, the TENM sequence can increase the expression level of a heterologous protein. In some embodiments, the TENM sequence can increase the expression level of a chimeric antigen receptor (CAR). In some embodiments, the expression level of a heterologous protein can be measured by the Median Fluorescence Intensity (MFI) ratio between CAR positive cells and CAR negative cells (MFI CAR positive/CAR negative).

In some embodiments, the TENM sequence can increase the expression level of a heterologous protein by about or at least 10%, 20%, 30%, 40%, 50%, 60%, 70%, 80%, 100%, 120%, 140%, 160%, 180%, 200%, 300%, 400%, 500%, 600%, or 700% (e.g., as compared to a control sequence that is different from the TENM sequence).

In some embodiments, the expression level (as measured by MFI ratio) is higher than 1, higher than 2.5, higher than 5, higher than 7.5, higher than 10, higher than 12.5, higher than 15, higher than 17.5, higher than 20, higher than 22.5, higher than 25, higher than 27.5, higher than 30, higher than 32.5, higher than 35, higher than 37.5, higher than 40, higher than 42.5, higher than 45, higher than 47.5, or higher than 50.

In some embodiments, the TENM sequence can be used to increase titer of virus vectors from packaging cells. In some embodiments, the packaging cells are HEK293T cells.

In some embodiments, the TENM sequence can be used to produce a specific titer of a lentiviral vector. The specific titer is determined in a test system utilizing a cell target (e.g., Jurkat cells) and lentiviral vector transduction in vitro, followed by analysis of transduced cells.

In some embodiments, titer is expressed as the number of cells that are positively transduced per ml as a result of transduction with a certain volume of virus particles. In some embodiments, titer is calculated using the below formula: Titer (TU/mL)=cell number×positive rate (%)/virus volume (mL).

In some embodiments, the titer is between 1×10⁵ and 1×10¹⁵ TU/mL, for example, between 1×10⁷ and 1×10¹³ TU/mL, or between 1×10⁹ and 1×10¹¹ TU/mL. In some embodiments, the titer is between 1×10⁷ and 1×10⁹ TU/mL. In some embodiments, the titer is higher than 1×10⁸ TU/mL, higher than 2×10⁸ TU/mL, higher than 3×10⁸ TU/mL, higher than 4×10⁸ TU/mL, higher than 5×10⁸ TU/mL, or higher than 6×10⁸ TU/mL.

In some embodiments, the regulatory sequence as described herein can increase the titer, e.g., by about or at least 10%, 20%, 30%, 40%, 50%, 60%, 70%, 80%, 100%, 120%, 140%, 160%, 180%, 200%, 300%, 400%, 500%, 600%, or 700% (e.g., as compared to a control regulatory sequence that is different from the TENM sequence).

In some embodiments, producing a specific titer of a lentiviral vector comprises adding a vector system to one or more cells. In some embodiments, the one or more cells is a cell line. In some embodiments, the cell line is a Jurkat cell line. In some embodiments, the cell line is a HeLa cell line. In some embodiments, the cell line is a CHO cell line. In some embodiments, the cell line is a Hep3B cell line. Persons skilled in the art will also appreciate that in other embodiments, the cell line may be any suitable cell line known in the art.

In some embodiments, the TENM sequence can increase the titer of an integrated lentiviral vector containing the TENM sequence by about or at least 10%, 20%, 30%, 40%, 50%, 60%, 70%, 80%, 100%, 120%, 140%, 160%, 180%, 200%, 300%, 400%, 500%, 600%, or 700% (e.g., as compared to a control sequence that is different from the TENM sequence).

### Protein Expression System

The “protein expression system” in the present disclosure refers to a system comprising a host and a vector containing a heterologous gene with one or more regulatory sequences or elements as described herein, and the purpose of the expression of the heterologous gene in the host can be achieved by this system. The protein expression system generally comprises the following parts: (1) a host, i.e., an organism expressing proteins, which may be selected from bacteria, yeast, plant cells, animal cells, human cells, and the like; (2) a vector. The type of the vector matches with the host. The vector contains a fragment of a heterologous gene. The heterologous gene can be expressed in the host via the mediation of the vector. In some embodiments, the expressed protein products are secreted. In some embodiments, the vectors are integrated into host cell DNA. In some embodiments, the vectors are delivered into the host cell using a virus.

Viruses are infectious agents composed of nucleic acids protected by a protein coat. These microbes cannot self-replicate, relying instead on the cells they infect to produce more copies of themselves by hijacking the host's replication machinery. Viruses are generally associated with disease, but genetic engineering has repurposed their biology to leverage their natural ability to modify or supplement the host cell genetic code, producing a new class of gene delivery tools known as viral vectors. Gamma retroviral (GRV) and lentiviral (LV) vectors are derived from enveloped RNA viruses of the retroviridae family and have been widely used in research and in the clinic due to their ability to integrate genetic material to the host cell genome and stably express a transgene, making them particularly suited to transduce highly replicating cells such as immune cells.

Gene delivery using modified retroviruses (retroviral vectors, e.g., lentiviral vectors) was developed. Sequences generally required in cis are the long terminal repeats (LTRs) and its vicinity: the inverted repeats (IR or att sites) required for integration, the packaging sequence T, the transport RNA-binding site (primer binding site, PBS), and some additional sequences involved in reverse transcription (the repeat R within the LTRs, and the polypurine tracts, PPT, necessary for plus strand initiation). To generate replication-defective retroviral vectors, the gag, pol, and env genes are generally entirely deleted and replaced with an expression cassette.

In some embodiments, the lentiviral vector comprises the regulatory sequence described herein, a gene of interest (GOI), a CMV promoter, a 5′ LTR, a packaging sequence T, and a 3′ LTR. In some embodiments, the lentiviral vector comprises, from the 5′ end to the 3′ end, a CMV promoter, a 5′ LTR, a packaging sequence T, the regulatory sequence described herein, a GOI, and a 3′ LTR. In some embodiments, to increase the transduction efficiency, the lentiviral vector can also contain a central polypurine tract (cPPT). The cPPT can support reverse transcription and potentially also nuclear translocation. In some embodiments, the lentiviral vector also contains the Rev-responsive element (RRE). The Rev/RRE interaction can overcome nuclear retention of the lentiviral genomic RNA, which is mediated by inhibitory sequences involving the splice donor and gag. In some embodiments, the lentiviral vector comprises, from the 5′ end to the 3′ end, a CMV promoter, a 5′ LTR, a packaging sequence T, a RRE, a cPPT, the regulatory sequence described herein, a GOI, and a 3′ LTR.

Lentiviruses are linked by similarities in genetic composition, molecular mechanisms of replication and biological interactions with their hosts. They are best known as agents of slow disease syndromes that begin insidiously after prolonged periods of subclinical infection and progress slowly; thus, they are referred to as the “slow” viruses. They have the same basic organization as all retroviruses, but are more complex due to the presence of accessory genes (e.g., vif, vpr, vpu, nef, tat, and rev), which play key roles in lentiviral replication in vivo.

Lentiviruses represent a genus of slow viruses of the Retroviridae family, which includes the human immunodeficiency viruses (HIV), the simian immunodeficiency virus (SIV), the equine infectious encephalitis virus (EIAV), the caprine arthritis encephalitis virus (CAEV), the bovine immunodeficiency virus (BIV) and the feline immunodeficiency virus (FIV). Lentiviruses can persist indefinitely in their hosts and replicate continuously at variable rates during the course of the lifelong infection. Persistent replication of the viruses in their hosts depends on their ability to circumvent host defenses.

The lentiviral particles, which contain lentiviral vectors, can be produced by recombinant technology upon transient transfection of human cultured cells (e.g., HEK 293T cells) by different DNA plasmids:
(i) packaging plasmids, which express at least the Gag, Pol, Rev and, in some cases, structural and/or enzymatic proteins necessary for the packaging of the transfer construct;
(ii) a transfer plasmid, containing an expression cassette and HIV cis-acting factors necessary for packaging, reverse transcription, and integration; and
(iii) an envelope-encoding plasmid, in most cases the glycoprotein of vesicular stomatitis virus (VSV.G), a protein that allows the formation of mixed particles (pseudotypes) that can target a wide variety of cells.

This procedure allows obtaining transient production of lentiviral particle vectors by the transfected cells. However, the lentiviral particle vectors may also be continuously produced by cells by stably inserting the packaging genes, the proviral coding DNA, and the envelope gene into the cellular genome. This allows the continuous production of lentiviral particle vectors by the cells without the need for transient transfection. Of course, a combination of these procedures can be used, with some of the DNAs/plasmids integrated into the cellular genome and others provided by transient transfection.

In some embodiments, the lentiviral vector comprises the regulatory sequence described herein, a gene of interest (GOI), a CMV promoter, a 5′ LTR, a packaging sequence T, and a 3′ LTR. In some embodiments, the lentiviral vector comprises, from the 5′ end to the 3′ end, a CMV promoter, a 5′ LTR, a packaging sequence T, the regulatory sequence described herein, a GOI, and a 3′ LTR. In some embodiments, the lentiviral vector further comprises a RRE and a cPPT. In some embodiments, the lentiviral vector comprises, from the 5′ end to the 3′ end, a CMV promoter, a 5′ LTR, a packaging sequence Ψ, a RRE, a cPPT, the regulatory sequence described herein, a GO1, and a 3′ LTR.

Enhancers are cis-acting sequences, which can act as transcriptional activators at a distance. They have been widely employed in viral derived vectors because they appear to be the most efficient for obtaining transgene strong expression in a variety of cell types. However, given the safety issue of insertional mutagenesis, such transcriptional enhancer sequences should be deleted from the lentiviral vector constructs to abolish the risk of insertional mutagenesis by enhancer proximity effect. This enhancer proximity effect is by far the most frequent mechanism of insertional mutagenesis and is the only effect described in human or animal cases of tumorigenic events after gene transfer. In some embodiments, the regulatory sequence described herein comprises an enhancer. In some embodiments, the regulatory sequence described herein does not comprise an enhancer.

### Vectors

The “vector” in the present disclosure refers to a delivery vehicle for a polynucleotide. In some embodiments, the vector includes a polynucleotide sequence encoding a certain protein operatively inserted therein and enables the expression of this protein in a genetic engineering recombinant technique. The vector can be used to transform, transduce or transfect a host cell, and enable the genetic material element carried by the vector to be expressed in the host cell. The “vector” in the present disclosure can be any suitable vector, which includes chromosomal, non-chromosomal and synthetic nucleic acid vectors (including a group of suitable nucleic acid sequences which express the control elements). Illustratively, said vector may be a recombinant plasmid vector, a recombinant eukaryotic viral vector, a recombinant phage vector, a recombinant yeast minichromosome vector, a recombinant bacterial artificial chromosome vector, or a recombinant yeast plasmid vector.

Illustratively, the vector in the present disclosure may include the derivatives of SV40, bacterial plasmids, phage DNAs, baculovirus, yeast plasmid, vectors derived from a combination of a plasmid and a phage DNA, and vectors such as virus nuclear acids (RNA or DNA). In some embodiments, the vector is an adeno-associated virus (AAV) vector. In some embodiments, the vector is a lentivirus vector. In a specific embodiment, the vector comprises one or more exemplary sequences that are shown in FIG. 1, FIG. 19 and FIG. 26.

The 5 main classes of viral vectors can be categorized in 2 groups according to whether their genomes integrate into host cellular chromatin (oncoretroviruses and LV) or persist in the cell nucleus predominantly as extrachromosomal episomes (AAV, AV, and herpes viruses). The choice of viral vectors for clinical use depends on the efficiency of transgene expression, ease of production, safety, toxicity, and stability. Furthermore, the types of vectors are represented by both RNA and DNA viruses with either single-stranded (ss) or double-stranded (ds) genomes.

The expression vector may additionally comprise one or more of the following: an origin of replication, a selectable marker, and a multiple cloning site.

In yet a further embodiment, the present disclosure provides an expression vector comprising the regulatory sequence described herein, wherein the regulatory sequence is operably-linked to a heterologous polynucleotide.

In some embodiments, the vector is used to deliver a gene of interest that encodes for a vaccine. In some embodiments, the vector is used to deliver a gene of interest that encodes for an antibody. In some embodiments, the vector is used to deliver a gene of interest that encodes for a chimeric antigen receptor (CAR). In some embodiments, the vector is used to deliver a gene of interest that encodes for a modified T-cell receptor (TCR).

In some embodiments, a vector is provided comprising the regulatory sequence described herein. In some embodiments, a vector is provided comprising the TENM sequence described herein. In some embodiments, a vector is provided comprising (1) a truncated hEF1α promoter, (2) a NF-κB binding site and (3) a MVM intron. In some embodiments, exemplary vector structures can be found in FIG. 1, FIG. 19 and FIG. 26.

In some embodiments, the vector is a viral vector. In some embodiments, a virus particle capable of infecting a target cell comprises an envelope protein optimized for infecting the target cell, and any of the vectors disclosed herein. In some embodiments, the target cell is a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, an immune cell (e.g., T cell or NK cell), or a precursor hematopoietic stem cell.

In some embodiments, the vector is a lentiviral vector. In some embodiments, the lentiviral vector is any of the lentiviral vectors comprising the regulatory sequence described herein. In some embodiments, the lentiviral vector is any of the lentiviral vectors comprising the TENM sequence or variants thereof. In some embodiments, the lentiviral vector is a lentiviral vector depicted in FIG. 1, FIG. 19 and FIG. 26.

In some embodiments, the lentiviral vector is comprised of an integrated lentiviral vector. In some embodiments, the integrated lentiviral vector is derived from a lentiviral vector system. In some embodiments, the lentiviral vector system comprises separate plasmids encoding a rev gene and an env gene. In some embodiments, the integrated lentiviral vector is derived from a 3-vector lentiviral system. In some embodiments, the integrated lentiviral vector is derived from a 4-vector lentiviral system.

### Lentivirus Vectors

Lentiviruses, a type of retrovirus (RV), consist of a single-stranded positive-sense RNA sequence that is transcribed into a DNA and integrated into the host genome, causing persistent infection. Most lentiviral vectors (LVVs) are derived from HIV-1 and retain the capability to integrate into the genome of infected cells. Wild-type HIV genome with all of its genes and regulatory elements provides the backbone for LVVs. In the first-generation LVVs, the envelope protein and the psi (ψ) packaging signal are incorporated into a heterologous promoter to reduce recombination potential. The second-generation LVVs have the accessory genes (vif, vpr, vpu, and nef) removed to reduce the virulence of any potential replication-competent retrovirus. Additionally, the third-generation LVVs have the transactivator gene, tat eliminated, which splits the vector into 3 plasmids to reduce further recombination potential, retaining only the 3 genes necessary for transgene expression (gag, pol, rev).

Lentiviral vectors have become very popular for clinical applications due to their ability to more efficiently transduce nonproliferating or slowly proliferating cells, such as CD34+ stem cells. Gene transfer using LVVs into CD34+ HSCs has been used to treat several genetic diseases, including β-thalassemia, X-linked adrenoleukodystrophy, metachromatic leukodystrophy, and Wiskott-Aldrich Syndrome. Third-generation, self-inactivating LVVs were also used to introduce genes into hematopoietic stem cells to correct primary immunodeficiencies and hemoglobinopathies. The LVVs have been used to alter T cells by introducing genes to generate immunity to fight cancer through the delivery of chimeric antigen receptors (CARs) or cloned T-cell receptors. CAR T-cell therapies developed using LVVs have been successful in the clinical setting in patients with B-cell malignancies. This has resulted in the regulatory approval of the first genetically engineered cellular therapy using these vector systems.

In some embodiments, the lentiviral vector is used to transfer a gene of interest that encodes for a chimeric antigen receptor (CAR), a modified T-cell receptor (TCR), an antibody, a fusion protein, an enzyme, a soluble protein, a membrane protein, a structural protein, a ribosome protein, a zymogen, a cell surface receptor protein, a transcriptional regulatory protein, a translational regulatory protein, a chromatin protein, a hormone, a cell cycle regulatory protein, a G protein, a neuroactive peptide, an immunomodulatory protein, a blood component protein, an ion gate protein, a heat shock protein, dihydrofolate reductase, an antibiotic resistance protein, a functional fragment of any one of the proteins, or any combination thereof.

As used in this disclosure, “lentiviral vector” or “lentivirus vector” means a non-replicating vector for the transduction of a host cell with a transgene comprising cis-acting lentiviral RNA or DNA sequences, and requiring lentiviral proteins (e.g., Gag, Pol, and/or Env) that are provided in trans. The lentiviral vector contains cis-acting packaging sequences, but lacks expression of functional Gag, Pol, and Env proteins. The lentiviral vector may be present in the form of an RNA or DNA molecule, depending on the stage of production or development of said retroviral vectors.

The lentiviral vector can be in the form of a recombinant DNA molecule, such as a plasmid. The lentiviral vector can be in the form of a lentiviral particle vector, such as an RNA molecule(s) within a complex of lentiviral and other proteins. Typically, lentiviral particle vectors, which correspond to modified or recombinant lentivirus particles, comprise a genome which is composed of two copies of single-stranded RNA. These RNA sequences can be obtained by transcription from a double-stranded DNA sequence inserted into a host cell genome (proviral vector DNA) or can be obtained from the transient expression of plasmid DNA (plasmid vector DNA) in a transformed host cell.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene. Efficient integration and replication in non-dividing cells generally requires the presence of two c/s-acting sequences at the center of the lentiviral genome, the central polypurine tract (cPPT) and the central termination sequence (CTS). These lead to the formation of a triple-stranded DNA structure called the central DNA “flap”, which acts as a signal for uncoating of the pre-integration complex at the nuclear pore and efficient importation of the expression cassette into the nucleus of non-dividing cells, such as dendritic cells.

Further sequences are usually present in cis, such as the long terminal repeats (LTRs) that are involved in integration of the vector proviral DNA sequence into a host cell genome. Vectors may be obtained by mutating the LTR sequences, for instance, in domain U3 of said LTR.

In one embodiment, the present disclosure provides a lentiviral vector comprising LTR sequences, optionally with a mutated U3 region removing promoter and enhancer sequences in the 3′ LTR.

The packaging sequence Ψ (psi) is incorporated to support encapsidation of the polynucleotide sequence into the vector particles.

In some embodiments, the lentiviral vector comprises the regulatory sequence, and an expression cassette including a transgene. In some embodiments, the lentiviral vector comprises (1) a truncated hEF1α promoter, (2) a NF-κB binding site and (3) a MVM intron, and an expression cassette including a transgene. In some embodiments, the lentiviral vector comprises (1) a truncated hEF1α promoter, (2) a NF-κB binding site and (3) a MVM intron, an expression cassette including a transgene, and one or more additional regulatory sequences (e.g., WPRE).

In some embodiments, the lentivirus vector does not contain a nucleic acid sequence from the lentiviral genome that expresses an envelope protein. In some embodiments, a separate vector that contains a nucleic acid sequence encoding an envelope protein operably linked to a promoter is used. This env vector also does not contain a lentiviral packaging sequence, in one embodiment the env nucleic acid sequence encodes a lentiviral envelope protein.

Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid. In another embodiment, the gag and pol genes are provided on a first plasmid and the rev gene is provided on a second plasmid. Accordingly, both 3-vector and 4-vector systems can be used to produce a lentivirus as described herein. In some embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line.

Various lentiviral packaging systems can be acquired commercially (e.g., Lenti-vpak packaging kit from OnGene Technologies, Inc., Rockville, MD), and can also be designed as described herein.

### Transgene

In one aspect, the present disclosure provides a vector (e.g., lentiviral vector) containing the regulatory sequence as described herein and a transgene or gene of interest (GOI).

In some embodiments, the transgene encodes a protein. In some embodiments, the vector serves to introduce this sequence into the transduced cell. In some embodiments, this sequence is integrated into the genome of the transduced cell. In some embodiments, the “transgene” or “gene of interest (GOI)” does not include those sequences of the vector that facilitate transduction of the transgene. The transgene may be a nucleic acid sequence from another organism. Alternatively, the transgene may be a nucleic acid sequence from the same organism, but having different regulatory sequences controlling its expression. The transgene may be a sense or antisense nucleic acid molecule. In some embodiments, the transgene is a heterologous polynucleotide.

In some embodiments, the transgene is a chimeric antigen receptor (CAR), a modified T-cell receptor (TCR), an antibody, a fusion protein, an enzyme, a soluble protein, a membrane protein, a structural protein, a ribosome protein, a zymogen, a cell surface receptor protein, a transcriptional regulatory protein, a translational regulatory protein, a chromatin protein, a hormone, a cell cycle regulatory protein, a G protein, a neuroactive peptide, an immunomodulatory protein, a blood component protein, an ion gate protein, a heat shock protein, dihydrofolate reductase, an antibiotic resistance protein, a functional fragment of any one of the proteins, and any combination thereof.

In some embodiments, the transgene sequence encodes a therapeutic protein. In some embodiments, the therapeutic protein is an antibody or antigen-binding fragment thereof. In some embodiments, the therapeutic protein is a chimeric antigen receptor (CAR). In some embodiments, the transgene comprises the sequence set forth in SEQ ID NO: 7. In some embodiments, the transgene sequence encodes an immunogenic polypeptide. In some embodiments, the immunogenic polypeptide is viral, parasitic, bacterial, or fungal. In one embodiment, the immunogenic polypeptide is a tumor antigen.

In some embodiments, the length of the transgene sequence is at least 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 10.5 kb, 11 kb, 11.5 kb, 12 kb, 12.5 kb, 13 kb, 13.5 kb, 14 kb, 14.5 kb, 15 kb, 15.5 kb, or 16 kb.

In some embodiments, the length of the transgene sequence is less than 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 10.5 kb, 11 kb, 11.5 kb, 12 kb, 12.5 kb, 13 kb, 13.5 kb, 14 kb, 14.5 kb, 15 kb, 15.5 kb, or 16 kb.

The present disclosure also provides a mammalian host cell comprising an expression vector described herein. The expression vector may be transfected into a host cell by any suitable method. Optionally, the host cell is a mammalian cell (e.g. a human cell), such as those mentioned above. Such host cells may be isolated cells.

The present disclosure further provides a mammal whose genome comprises a regulatory sequence described herein or an expression vector described herein. Optionally, the regulatory sequence or an expression vector is inserted into the genome of the mammal in such a way that a heterologous polynucleotide which is operably linked to said regulatory sequence or which is operably inserted into said expression vector is expressed in one or more cells of the mammal. In some embodiment, the mammal is a mouse or rat. In some embodiments, the mammal is a non-human mammal.

The present disclosure also provides methods of enhancing the expression of a recombinant protein or polypeptide. The methods involve inserting one or more vectors as described herein into cells (e.g., by transformation or transfection); culturing the cells in the medium; and recovering the recombinant protein or polypeptide so expressed.

### Methods of Treatment

The regulatory sequence, vector (e.g., lentivirus vector), virus particle, or a cell containing the regulatory sequence described herein can be used for various therapeutic purposes.

In some embodiments, the regulatory sequence described herein is used to express a therapeutic transgene encoding a polypeptide that provides curative, preventative, or ameliorative benefits to a subject in need thereof. In some embodiments, a virus containing the regulatory sequence described herein can infect and transduce the cell in vivo, ex vivo, or in vitro. In ex vivo and in vitro embodiments, the transduced cells can then be administered to a subject in need thereof. In some embodiments, the present disclosure is related to a method of using the vector described herein, virus particles described herein, and transduced cells described herein to treat, prevent, and/or ameliorate a disease.

In some embodiments, regulatory sequence, vector (e.g., lentivirus vector), virus particle, or a cell containing the regulatory sequence described herein can be used in gene therapy. In some embodiments, the gene therapy can be used to treat a genetic disorder. In some embodiments, the gene therapy can be used to treat lipoprotein lipase deficiency, large B-cell lymphoma, beta thalassemia, mantle cell lymphoma, acute lymphoblastic leukemia, vascular endothelial growth factor peripheral artery disease, multiple myeloma, cerebral adrenoleukodystrophy, hemophilia B, head and neck squamous cell carcinoma, multiple myeloma, bladder cancer, spinal muscular atrophy, adenosine deaminase deficiency (ADA-SCID), melanoma, B cell lymphoblastic leukemia, hemophilia A, or Leber congenital amaurosis.

In some embodiments, the vector containing the regulatory sequence can be administered by direct injection to a cell, tissue, or organ of a subject, in vivo. In various other embodiments, cells are transduced in vitro or ex vivo with vectors described herein. The transduced cells are then administered to a subject.

Cells suitable for transduction and administration include, but are not limited to stem cells, progenitor cells, and differentiated cells including immune cells (e.g., T cells, NK cells). In certain embodiments, the transduced cells are T cells. In some embodiments, the cells are derived from the patient. In some embodiments, the cells are derived from a healthy donor.

The transduced T cells contemplated herein provide improved methods of adoptive immunotherapy for use in the treatment of various tumors and cancers. In particular embodiments, the specificity of a primary T cell is redirected to tumor or cancer cells by genetically modifying the primary T cell with a CAR contemplated herein. In various embodiments, a viral vector is used to genetically modify an immune effector cell with a polynucleotide comprising the regulatory sequence described herein and encoding a CAR.

In one embodiment, the present disclosure is related to a type of cellular therapy where T cells are genetically modified to express a CAR that targets cancer cells that express a target antigen, and the CAR T cell is infused to a recipient in need thereof. The infused cell is able to kill tumor cells in the recipient. In some embodiments, CAR T cells are able to replicate in vivo resulting in long-term persistence that can lead to sustained cancer therapy.

In some embodiments, compositions comprising an immune effector cell genetically modified with a vector comprising the regulatory sequence disclosed herein operably linked to a polynucleotide encoding a CAR are used in the treatment of solid tumors or cancers including, without limitation, liver cancer, pancreatic cancer, lung cancer, breast cancer, bladder cancer, brain cancer, bone cancer, thyroid cancer, kidney cancer, or skin cancer.

In some embodiments, methods comprising administering a therapeutically effective amount of CAR-expressing immune effector cells contemplated herein or a composition comprising the same, to a patient in need thereof, alone or in combination with one or more therapeutic agents, are provided. In certain embodiments, the cells described herein are used in the treatment of patients at risk for developing a cancer. Thus, the present disclosure provides methods for the treatment or prevention of a cancer comprising administering to a subject in need thereof, a therapeutically effective amount of the CAR-modified T cells of the disclosure.

In some embodiments, transduced cells can be stimulated with cytokines for expansion. In some embodiments, subjects are administered 1, 2, 3, 4, 5, or more doses over days, months, or years, as needed to maintain or increase the expression of therapeutic proteins.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In some embodiments, one or more additional therapeutic agents can be administered to the subject. In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

### EXAMPLES

The disclosure is further described in the following examples, which do not limit the scope of the disclosure described in the claims.

### Example 1: Construction of Lentiviral Vector pCsin-hEF1αΔ

The backbone designs of the lentiviral vectors used in the examples are shown in FIG. 1.

Based on the typical third-generation lentiviral vector pCsin-hEF1α, the full-length EF1α promoter (SEQ ID NO: 5, NCBI No: AL603910.6, hereinafter referred to as EF1α promoter) was replaced with the EF1α promoter without intron sequences (SEQ ID NO: 6, hereinafter referred to as the short EF1α promoter) to obtain the lentiviral vector pCsin-hEF1αS. Based on pCsin-hEF1α lentiviral vector, the full-length EF1α promoter was replaced by EF1αΔ407-757 promoter (SEQ ID NO: 2) to obtain the lentiviral vector pCsin-hEF1αΔ. The lentiviral vectors constructed above were subjected to plasmid extraction for subsequent testing. In this example, a third-generation lentiviral packaging system was used. In addition to the above plasmids, pMDLg/pRRE plasmid encoding gag/pol, pMD2.G plasmid encoding VSV-G (Vesicular stomatitis virus G) envelope protein, and pRSV-Rev plasmid encoding REV protein were also included. HEK293T (ATCC) cells were used as packaging cells, and transient transfection of plasmids was used in the packaging process. The specific steps of lentivirus preparation are as follows:

6×10⁶ HEK293T cells in good growth state were plated into a T75 culture flask. 15 mL DMEM complete medium was added to culture the cells for about 20 hours until the confluence reached 80-90%. Lentiviral packaging plasmids including pMDLg/pRRE, pRSV-Rev and pMD2.G plasmids, were used to co-transfect HEK293T cells with PEIpro-HQ. 4-6 hours after transfection, the medium was replaced with DMEM complete medium. After 48 hours, the virus-containing supernatant culture medium was collected, centrifuged at 4,000 g for 10 minutes to obtain the supernatant containing the virus. After adding PEG6000 (⅓ of the supernatant volume), the supernatant was incubated overnight in a 4 °C refrigerator, and centrifuged at 3,000 g for 30 minutes to obtain a virus pellet. The pellet was re-suspended with DPBS solution to obtain a concentrated virus particle solution.

In this example, the target gene is CAR #1 (SEQ ID NO: 7). The cells after lentiviral transduction were detected by flow cytometry, and the titer was calculated according to the following formula:

Titer (TU/mL)=cell number×positive rate (%)/virus volume (mL)

Specifically, Jurkat cells (ATCC) in exponential growth phase were centrifuged and counted. The cell density was adjusted to 1×10⁶ cells/mL. The cells were plated in a sterile 96-well flat-bottom plate at 50 μL/well. Serially diluted virus vectors (3-fold gradient dilution) were added to each well of the 96-well plate. The cells were cultured for 72 hours in an incubator. The cells in each well were collected and the detection antibody (Anti-camel sdAb pAb Alexa Fluor 488) was added. After further incubation and washing, the cells were analyzed by flow cytometry to determine the proportion of positive cells. Wells with 5-20% positive rates and showing a linear relationship to virus vector concentration were selected, and the virus titers were calculated according to the above formula.

To determine expression levels (measured by MFI), commercial PBMC preparations from healthy donors (Shanghai Saili Biotechnology Co., Ltd.) were resuscitated in a blood warming instrument, and magnetic beads (Miltenyi Biotec) with a T cell antibody were added to pull down T cells. T cells were re-suspended in complete TexMACs medium (Miltenyi Biotec), and activated by adding an activation reagent (Miltenyi Biotec). T cells were centrifuged and counted. The cell density was adjusted to 1×10⁶ cells/mL. The cells were plated in a sterile 96-well flat-bottom plate at 50 μL/well. Serially diluted virus vectors (6-fold gradient dilution) were added to each well of the 96-well plate. The cells were cultured for 72 hours in an incubator. The cells in each well were collected and the detection antibody (Anti-camel sdAb pAb Alexa Fluor 488) was added. After further incubation and washing, the cells were analyzed by flow cytometry to determine the proportion of positive cells. The Median Fluorescence Intensity (MFI) of positive cells and negative cells was detected to calculate the MFI ratio between CAR positive cells and CAR negative cells (MFI CAR positive/CAR negative).

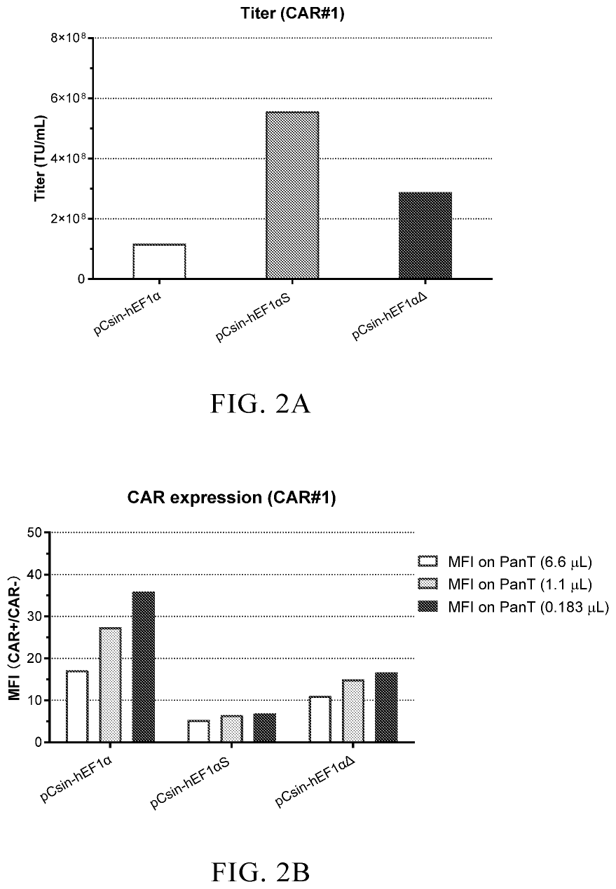

The titer and expression level (as measured by MFI) of the optimized lentiviral vectors in this example are shown in FIGS. 2A-2B. The titers of lentiviral vectors pCsin-hEF1α, pCsin-hEF1αS and pCsin-hEF1αΔ were 1.17×10⁸, 5.56×10⁸ and 2.88×10⁸ Tu/ml, respectively. As shown in FIGS. 2A-2B, compared with pCsin-hEF1α, although the pCsin-hEF1αS vector can effectively increase the titer of the lentivirus, the expression level of the target gene was significantly reduced. The EF1αΔ407-757 promoter and the pCsin-hEF1αΔ vector can also effectively increase the virus titer (2.46 folds), but the MFI decreased to some extent.

### Example 2: Construction of Lentiviral Vector pCsin-hEF1αΔ.Mi

As shown in FIG. 1, based on the pCsin-hEF1αA lentiviral vector, the MVM intron (small intron region of the minute virus of mice) sequence (SEQ ID NO: 3) was inserted at the 3′ end of the EF1αΔ407-757 promoter to obtain the lentiviral vector pCsin-hEF1αΔ.Mi. In this example, the target gene is also CAR #1. The methods in Example 1 were used for lentivirus packaging, positive cell detection, titer determination and expression level determination.

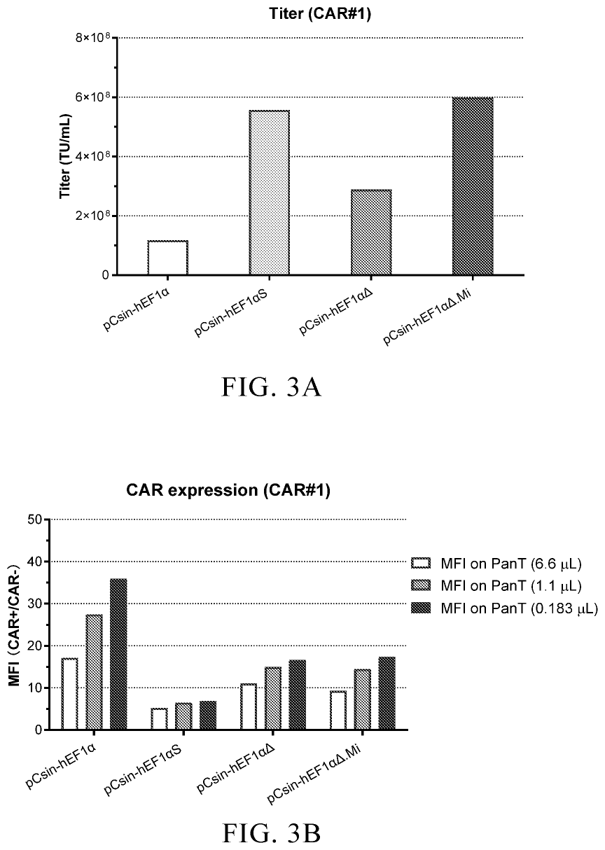

The titer and protein expression levels (as measured by MFI) of the modified lentiviral vector in this example are shown in FIGS. 3A-3B. The titers of lentiviral vectors pCsin-hEF1α, pCsin-hEF1αS, pCsin-hEF1αΔ and pCsin-hEF1αΔ.Mi were 1.17×10⁸, 5.56×10⁸, 2.88×10⁸ and 5.99×10⁸ Tu/ml, respectively. As shown in FIGS. 3A-3B, compared with pCsin-hEF1α, the MVM intron and the modified pCsin-hEF1αΔ.Mi vector significantly increased the titer of the lentivirus (5.12 folds). Compared with pCsin-hEF1αΔ, the addition of the MVM intron had little effect on the expression level of the target gene.

### Example 3: Construction of Lentiviral Vectors pCsin-NB.hEF1α, pCsin-NB.hEF1αS and pCsin-NB.hEF1αΔ.Mi

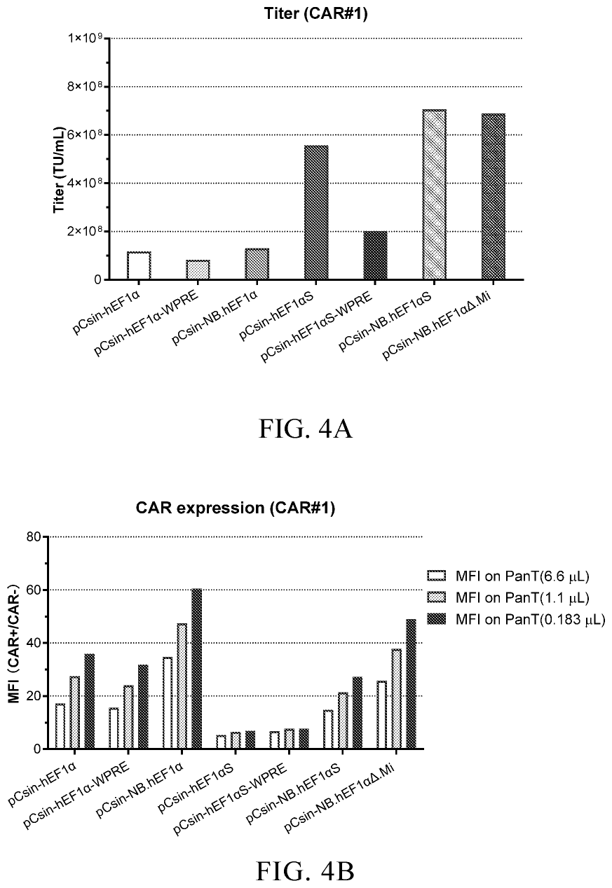

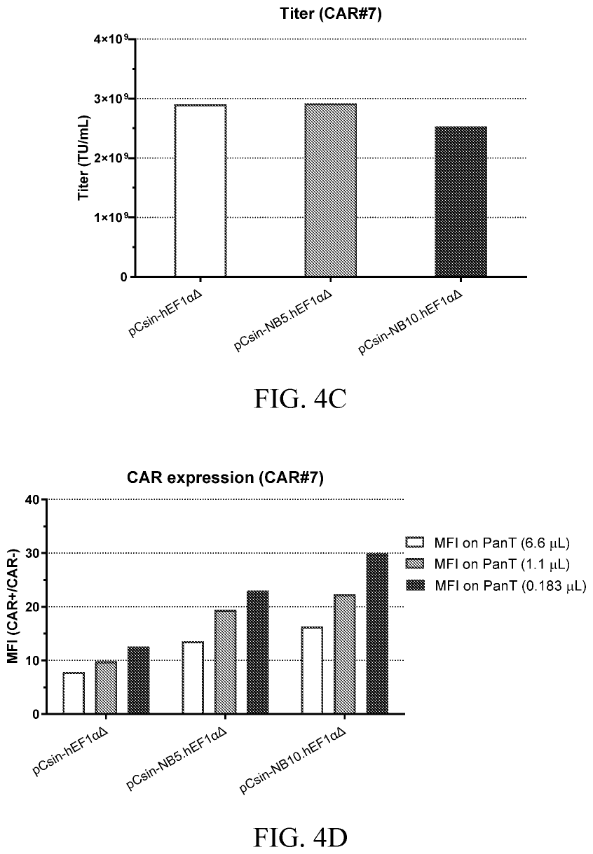

As shown in FIG. 1, based on the pCsin-hEF1α, pCsin-hEF1αS and pCsin-hEF1αΔ.Mi vectors, 5 copies of the NF-κB transcription factor binding sequence (SEQ ID NO: 1) and 7 bp connecting or linker sequence (AGCTCCT) was inserted at the 5′ end of the EF1α promoter to construct pCsin-NB.hEF1α, pCsin-NB.hEF1αS and pCsin-NB.hEF1αΔ.Mi vectors. At the same time, based on pCsin-hEF1α and pCsin-hEF1αS vectors, WPRE elements (SEQ ID NO: 12) were added to construct pCsin-hEF1α-WPRE and pCsin-hEF1αS-WPRE vectors. In this example, the target genes include CAR #1 and CAR #7 (2,661 bp). The methods in Example 1 were used for lentivirus packaging, positive cell detection, titer determination and expression level determination. As shown in FIG. 4A, the titers of lentiviral vectors pCsin-hEF1α, pCsin-hEF1α-WPRE, pCsin-NB.hEF1α, pCsin-hEF1αS, pCsin-hEF1αS-WPRE, pCsin-NB.hEF1αS and pCsin-NB.hEF1αΔ.Mi were 1.17×10⁸, 8.22×10⁷, 1.29×10⁸, 5.56×10⁸, 2.01×10⁸, 7.06×10⁸, and 6.88×10⁸ Tu/ml, respectively.

In some cases, adding WPRE elements can increase the expression of target genes in lentiviral vectors. However, as shown in FIGS. 4A-4B, the introduction of the WPRE element (as shown in pCsin-hEF1α-WPRE and pCsin-hEF1αS-WPRE vectors) reduced the titer of the lentivirus, and there was no significant impact on expression levels. As shown in FIG. 4B, introduction of the NF-κB transcription factor binding sequences (as shown in the pCsin-NB.hEF1α, pCsin-NB.hEF1αΔ and pCsin-NB.hEF1αΔ.Mi vectors) can stably increase the expression level of the target gene.

Additionally, constructs containing 5 copies of the NF-κB binding site (pCsin-NB5.hEF1αΔ) and constructs containing 10 copies of the NF-κB binding site (pCsin-NB10.hEF1αΔ) were constructed and tested. The results showed that higher copy numbers led to a higher expression level and similar titers (FIGS. 4C-4D).

### Example 4: Construction of Lentiviral Vectors pCsin-NB.hEF1αΔ.Mi, pCsin-NB.Mi.hEF1αΔ and pCsin-Mi.NB.hEF1αΔ

In Examples 1-3, the functions of the EF1αΔ407-757 promoter, the MVM intron, and the NF-κB transcription factor binding sequence in the optimization of the lentiviral vector have been respectively verified. In this example, the above elements were combined to construct different chimeric promoters. As shown in FIG. 1, the following vectors were designed by rearranging the positions of the elements: pCsin-NB.hEF1αΔ.Mi, pCsin-NB.Mi.hEF1αΔ and pCsin-Mi.NB.hEF1αΔ.

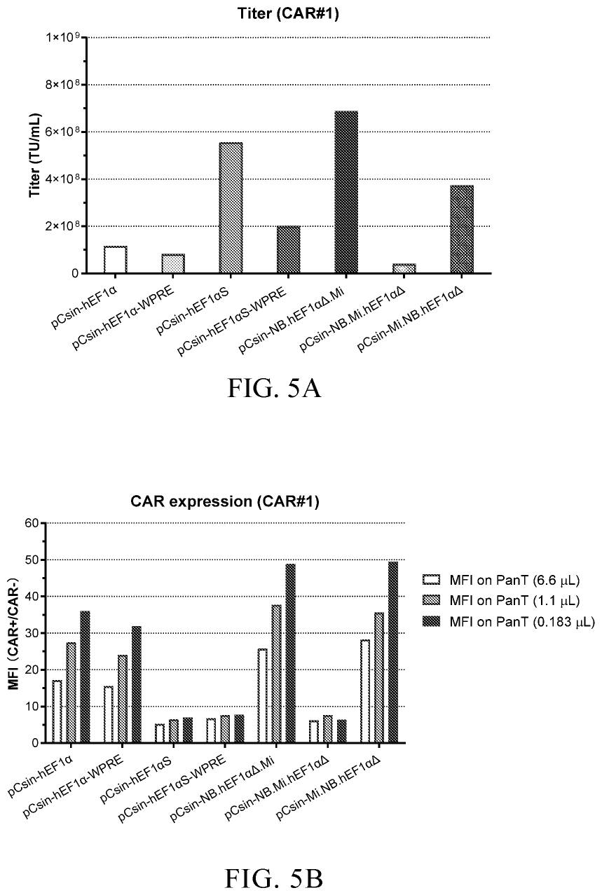

In this example, the target gene is also CAR #1. The methods in Example 1 were used for lentivirus packaging, positive cell detection, titer determination and expression level determination. Viral titers of lentiviral vectors pCsin-hEF1α, pCsin-hEF1α-WPRE, pCsin-hEF1αS, pCsin-hEF1αS-WPRE, pCsin-NB.hEF1αΔ.Mi, pCsin-NB.Mi.hEF1αA and pCsin-Mi.NB.hEF1αA were 1.17×10⁸, 8.22×10⁷, 5.56×10⁸, 2.01×10⁸, 6.88×10⁸, 4.03×10⁷ and 3.73×10⁸ Tu/ml, respectively (FIG. 5A). As shown in FIGS. 5A-5B, pCsin-NB.hEF1αΔ.Mi and pCsin-Mi.NB.hEF1αΔ both showed high expression levels, and the virus titers were significantly better than that of the pCsin-hEF1α vector. Notably, although lentiviral vectors pCsin-hEF1αS and pCsin-hEF1αS-WPRE also displayed high viral titers, the two vectors were of no practical use because of the extremely low expression level of target genes (FIG. 5B). Among them, the chimeric TENM promoter (truncated hEF1α promoter armored with NF-κB binding site and MVM intron) containing the NF-κB transcription factor binding sequence, the EF1αΔ407-757 promoter and the MVM intron arranged in the 5′-3′ order (SEQ ID NO: 4) and the corresponding lentiviral vector pCsin-NB.hEF1αΔ.Mi performed unexpectedly well in terms of virus titer and target gene expression.

### Example 5: Virus Titer and Target Gene Expression Performance of Lentiviral Vector pCsin-NB.hEF1αΔ.Mi

In Example 4, the performance of lentiviral titers and expression level of lentiviral vector pCsin-NB.hEF1αΔ.Mi have been well verified. In this example, the preferred lentiviral vector pCsin-NB.hEF1αΔ.Mi was tested with more target genes (CAR #3, 3,771 bp; CAR #7; CAR #8, 3,789 bp). The methods in Example 1 were used for lentivirus packaging, positive cell detection, titer determination and expression level determination. Besides the titers of concentrated viral suspension, the titers of viral supernatant were also determined.

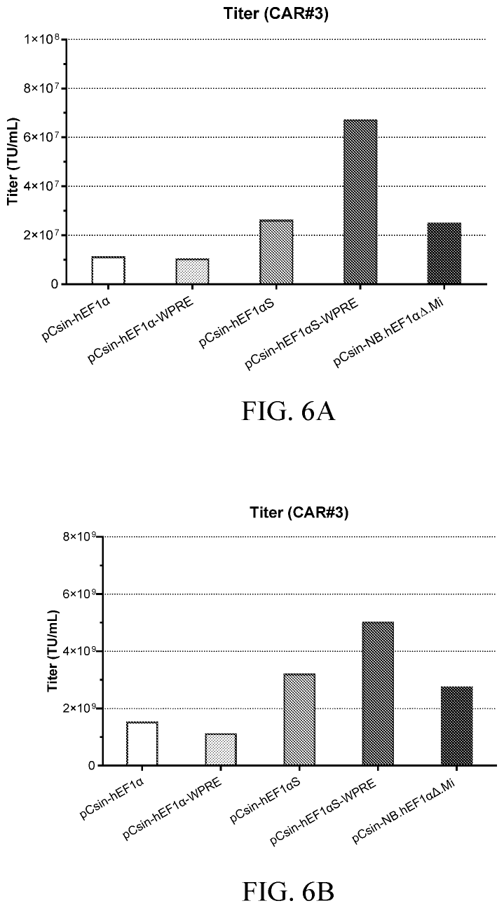

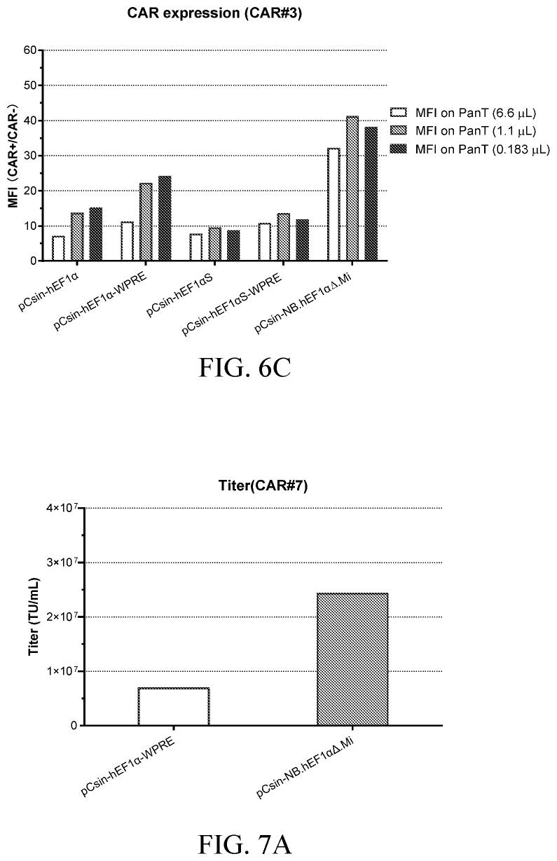

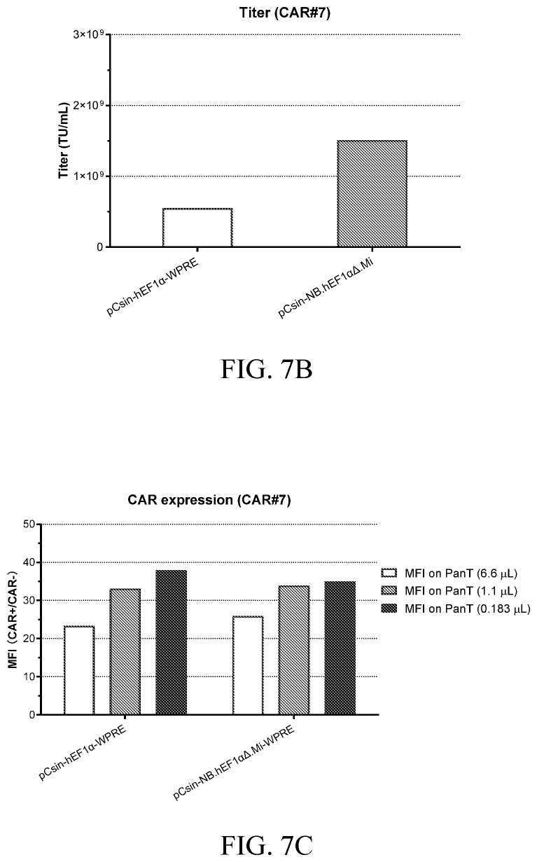

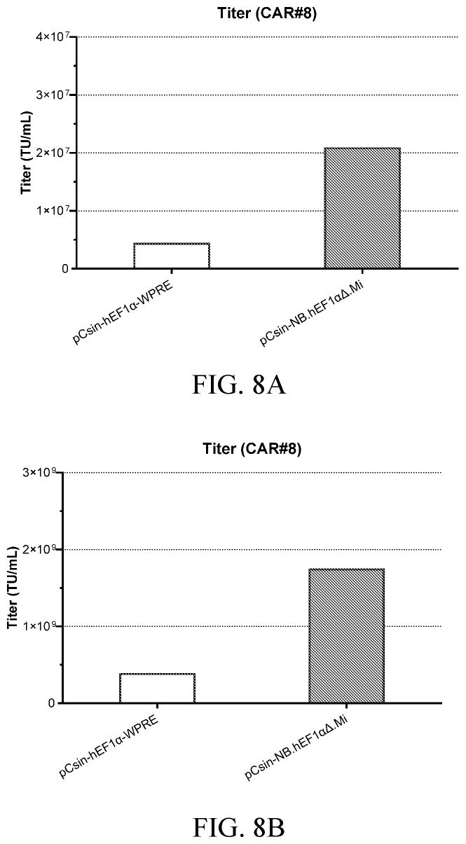

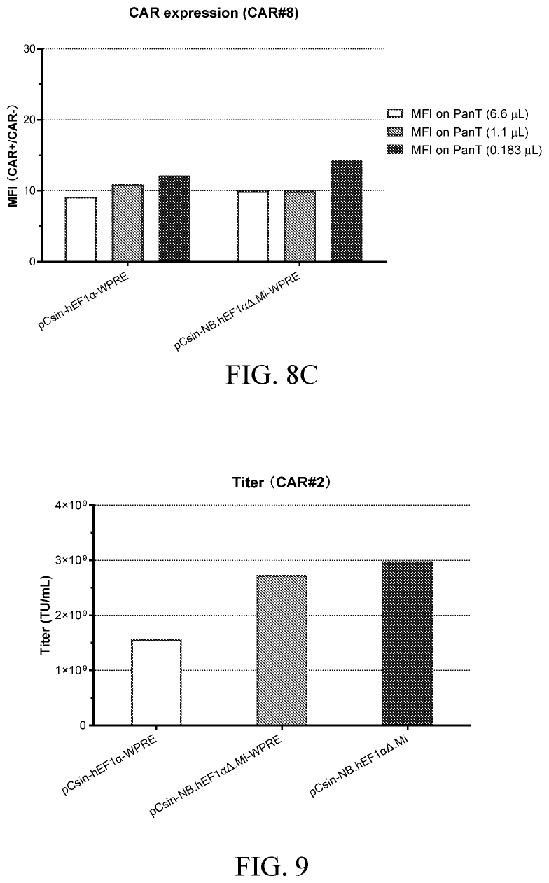

As shown in FIG. 6A, FIG. 7A, and FIG. 8A, for CAR #3, CAR #7 and CAR #8, the virus supernatant titers of lentiviral vector pCsin-hEF1α-WPRE were 1.05×10⁷, 7.03×10⁶ and 4.43×10⁶ Tu/ml, respectively. Parallelly, the virus supernatant titers of lentiviral vector pCsin-NB.hEF1αΔ.Mi were 2.50×10⁷, 2.44×10⁷, and 2.09×10⁷ Tu/ml.

As compared with pCsin-hEF1α, the TENM promoter and novel lentiviral vector pCsin-NB.hEF1αΔ.Mi reached 2.38, 3.47 and 4.72 folds increase in titers of the lentivirus. As shown in FIG. 6B, FIG. 7B, and FIG. 8B, for CAR #3, CAR #7 and CAR #8, the concentrated virus titers of lentiviral vector pCsin-hEF1α-WPRE were 1.13×10⁹, 5.49×10⁸ and 3.92×10⁸ Tu/ml, respectively. Parallelly, the concentrated virus titers of lentiviral vector pCsin-NB.hEF1αΔ.Mi were 2.77×10⁹, 1.51×10⁹, 1.75×10⁹ Tu/ml, which improve viral titers to 2.45, 2.75 and 4.46 folds, respectively. Consistent with the results shown in Examples 3 and 4, lentiviral vector pCsin-NB.hEF1αΔ.Mi maintained the related high expression level of target genes (FIG. 6C, FIG. 7C and FIG. 8C). In this example, lentiviral vector pCsin-NB.hEF1αΔ.Mi demonstrated the robust titer promotion and universality with different target genes, and the virus concentration did not diminish the effect. Meanwhile, the EF1α promoter and the WPRE element constitute 1,927 bp of the vector, and the preferred sequence length of the TENM promoter (e.g. NB.hEF1αΔ.Mi, containing 5 copies of NK-kB binding site) is only 1,153 bp. Therefore, the TENM promoter and the optimized lentiviral vectors can not only effectively increase the packaging titer of the lentivirus while maintaining or increasing the expression level of the target gene, but also provide a larger packaging capacity for the target gene.

### Example 6: In Vitro Long-Term Killing Assay of CAR-T Cells with Lentiviral Vectors, pCsin-hEF1α-WPRE, pCsin-NB.hEF1αΔ.Mi and pCsin-NB.hEF1αΔ.Mi-WPRE

In this example, an in vitro long term killing assay was performed to evaluate the efficacy performance of the optimized lentiviral vectors pCsin-NB.hEF1αΔ.Mi and pCsin-NB.hEF1αΔ.Mi-WPRE.

The methods in Example 1 were used for lentivirus packaging, positive cell detection, titer determination and expression level determination. In this example, the target gene is CAR #2 (2,187 bp). To produce CAR-T cells, commercial PBMC preparations from two healthy donors were resuscitated in a blood warming instrument, and magnetic beads with a T cell antibody were added to sort T cells. After sorting, T cells were activated by antibodies conjugated beads. CAR-T cells were produced with lentiviral vectors pCsin-hEF1α-WPRE, pCsin-NB.hEF1αΔ.Mi and pCsin-NB.hEF1αΔ.Mi-WPRE at MOI (multiplicity of infection)=4. CAR-T cells and target cell SHP-77 were co-incubated at ratio of E:T (Effect:Target)=2:1, and tumor cell residual was detected by flow cytometry every 1-2 days. A new round of incubation and killing assay was performed until tumor residual was higher than 20%, and then the serial killing assay was terminated. At the conclusion study, percentages of effect T cells (Teff, CD3+CD45RA+CCR7−), effect memory T cells (Tem, CD3+CD45RA−CCR7−), central memory T cells (Tcm, CD3+CD45RA−CCR7+) and naïve T cells (Tn, CD3+CD45RA+CCR7+) were detected by flow cytometry to evaluate the influence of lentiviral vectors on T cell phenotype.

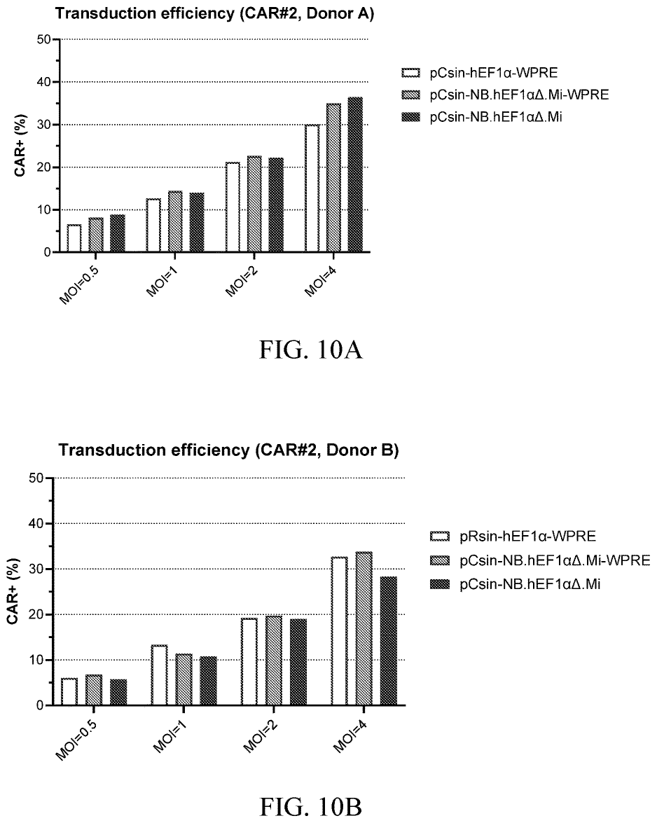

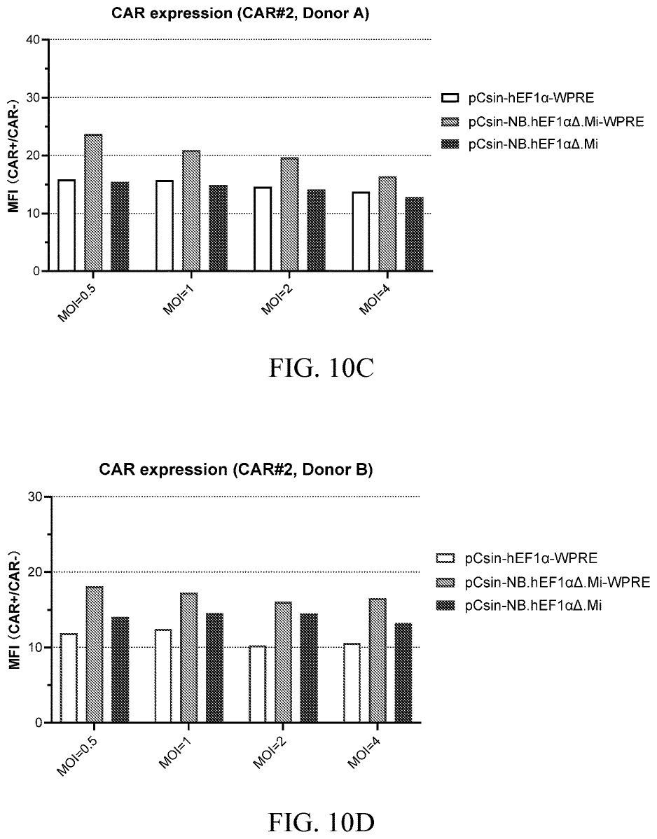

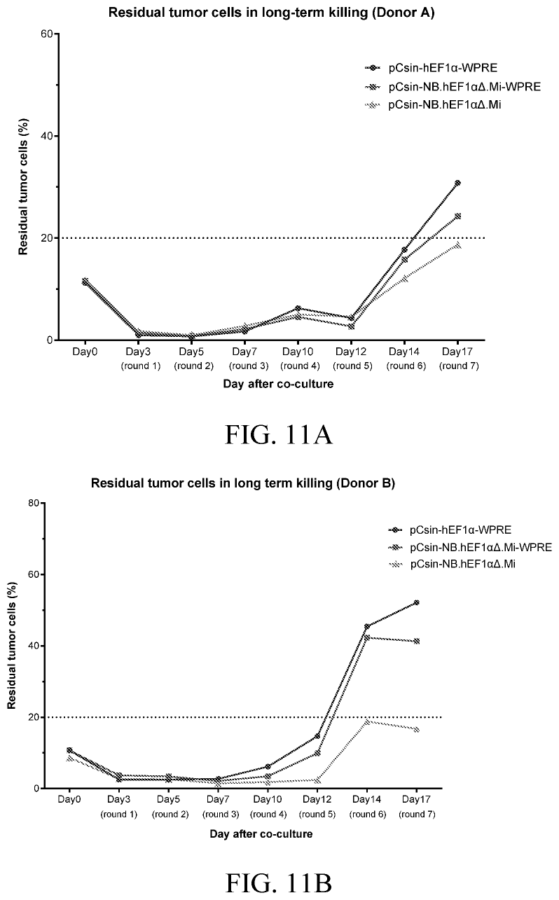

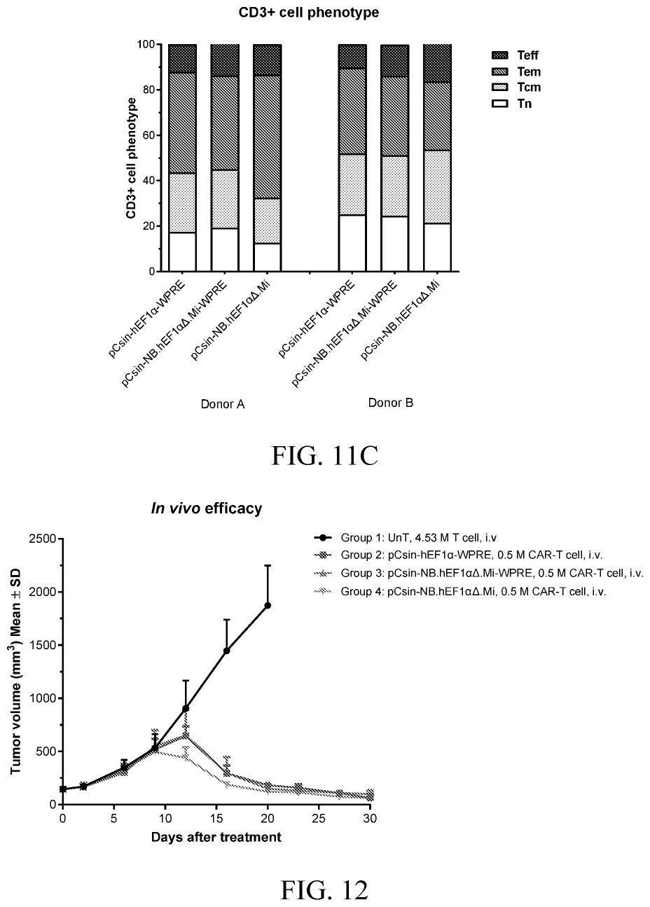

As shown in FIG. 9, the optimized lentiviral vectors pCsin-NB.hEF1αΔ.Mi-WPRE and pCsin-NB.hEF1αΔ.Mi displayed higher titers than that of pCsin-hEF1α-WPRE (1.75 and 1.91 folds). Lentiviral vector of pCsin-NB.hEF1αΔ.Mi did not show dramatical promotion because the titer of CAR #2 were naturally higher than other CARs used in some embodiments. The transduction efficiency and expression level of CAR #2 were as expected in preparation process with two donors (FIGS. 10A-10D). The in vitro serial killing results of the optimized lentiviral vectors in this example are shown in FIGS. 11A-11B. As compared with Group 1 with lentiviral vector pCsin-hEF1α-WPRE, CAR-T cells produced using lentiviral vectors pCsin-NB.hEF1αΔ.Mi and pCsin-NB.hEF1αΔ.Mi-WPRE displayed equivalent efficacy at E:T ratio=2:1. For both Donor A and Donor B, the CAR-T cells transduced with lentiviral vector pCsin-NB.hEF1αΔ.Mi even displayed slightly better efficacy in 7th round (percentages of tumor residual<20%), and the WPRE element did not show the necessity for the optimized lentiviral vectors. The data of FIG. 11C indicated slight difference of CD3+ cell phenotype among groups of lentiviral vectors pCsin-hEF1α-WPRE, pCsin-NB.hEF1αΔ.Mi and pCsin-NB.hEF1αΔ.Mi-WPRE. The percentages of effect T cells (Teff), effect memory T cells (Tem), century memory T cells (Tcm), and naïve T cells (Tn) indicated that the optimization of lentiviral vectors did not alter phenotype of T cells.

### Example 7: In Vivo Efficacy Study of CAR-T Cells with Lentiviral Vectors pCsin-hEF1α-WPRE, pCsin-NB.hEF1αΔ.Mi and pCsin-NB.hEF1αΔ.Mi-WPRE

In this example, an efficacy study in animal model was performed to evaluate the in vivo performance of the optimized lentiviral vectors pCsin-NB.hEF1αΔ.Mi and pCsin-NB.hEF1αΔ.Mi-WPRE.

In this example, the target gene is CAR #2. NOD-Prkdc^{em26Cd52}Il2rg^{em26Cd22}/NjuCrl (NCG) mice were raised in specific pathogen free (SPF) level facility after one week of quarantine. SHP-77 cell suspension was injected subcutaneously at the dose of 1×10⁷ cells per mouse. Twenty (20) days post inoculation, 20 mice were selected and randomized into 4 groups, with the mean tumor volume reached 145 mm³. After cell counting, 0.5 million CAR-T cells or 4.53 million mock T cells (adjusted according to CAR+%) were injected via tail vein (Day 0). Mice were observed every day and tumor volume were monitored three times a week. Once the mice lost more than 20% of body weight, or the tumor volume reached 2,000 mm³, the mice were euthanized to guarantee the animal welfare. The subcutaneous tumor volume was calculated according to the following formula:

Tumor volume (mm³)=½×tumor long diameter (mm)×tumor short diameter (mm)²

As shown in FIG. 12, mock T cells did not display apparent efficacy during the study (904.71±262.33 mm³), and the tumor volume of Group 1 increased rapidly. Other groups showed observable tumor inhibition effect on Day 13 (645.85±91.51 mm³, 652.02±241.62 mm³ and 440.24±979.4 mm³), and compared with Group 2 with lentiviral vector pCsin-hEF1α-WPRE, Group 4 with lentiviral vector pCsin-NB.hEF1αΔ.Mi showed better tumor inhibition efficacy (P value=0.0090). As the same as CAR-T cells generated using lentiviral vector pCsin-hEF1α-WPRE, the CAR-T cells with optimized lentiviral vectors pCsin-NB.hEF1αΔ.Mi and pCsin-NB.hEF1αΔ.Mi-WPRE could effectively eliminate the target cells during the course of treatment, and the WPRE element did not show the necessity for the optimized lentiviral vectors. At the conclusion of this study, all groups except Group 1 were almost tumor free (63.10±39.30 mm³, 98.15±35.22 mm³ and 61.82±31.42 mm³), and CAR-T cells with lentiviral vectors pCsin-hEF1α-WPRE, pCsin-NB.hEF1αΔ.Mi and pCsin-NB.hEF1αΔ.Mi-WPRE displayed equivalent and robust in vivo efficacy without observable toxicity.

### Example 8: Transcription Level Post-Activation of Lentiviral Vectors pCsin-hEF1α-WPRE and pCsin-NB.hEF1αΔ.Mi-WPRE

WPRE element was widely reported to enhance the stability of mRNA and elevate the expression of target genes. To exclude the influence of post-transcriptional regulation, and accurately determine the transcription level of the TENM promoter, we compared the transcript levels of lentiviral vectors pCsin-hEF1α-WPRE and pCsin-NB.hEF1αΔ.Mi-WPRE. In this example, the target gene is CAR #2.

CAR-T cells were prepared as description in Example 1. SHP-77 tumor cells were cultured in RPMI 1640 medium containing 10% FBS (fetal bovine serum). After the measurement of cell concentration, CAR-T cells were diluted to 0.8 million CAR positive cells per milliliter (adjusted according to CAR+%). Likewise, tumor cells were diluted to 0.8 million per milliliter. One mL of diluted tumor cells and 1 mL of diluted CAR-T cells were pipetted into each well of a 24-well plate in sequence and mixed well, reaching a E:T=1:1 ratio. Two days post co-incubation, cells were collected and analyzed by flow cytometry for CD3+% and CAR+%, while the rest underwent RNA extraction. RNA to cDNA transfer was done using PrimeScript RT Reagent Kit and then a qPCR assay was performed to evaluate the mRNA level. Specifically, each sample was detected by a pair of CAR-specific primers in triplicate with GAPDH used as an internal control, and relative fold change was analyzed based on the ΔΔCt method.

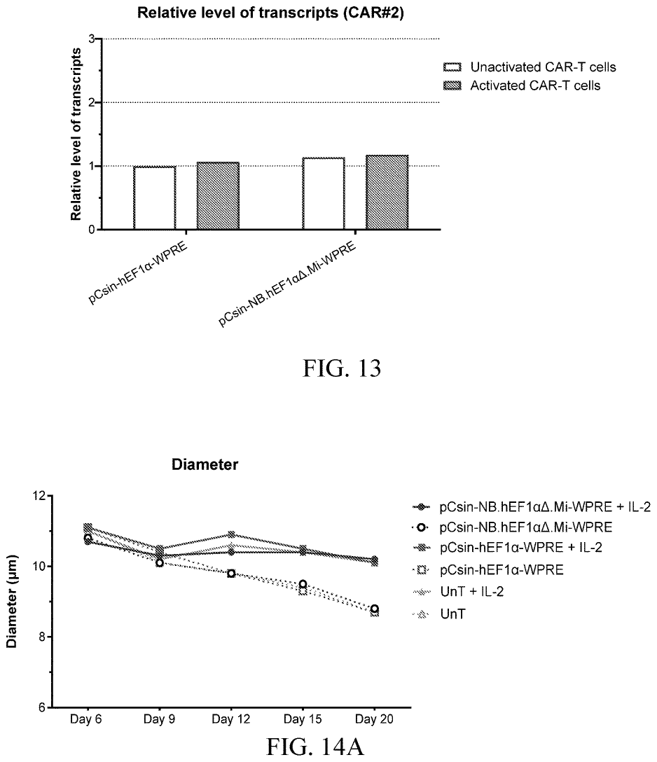

As shown in FIG. 13, both lentiviral vectors pCsin-hEF1α-WPRE and pCsin-NB.hEF1αΔ.Mi-WPRE showed similar related transcript level (1.00 and 1.06) without activation by target cells. The related transcript level of lentiviral vectors pCsin-hEF1α-WPRE and pCsin-NB.hEF1αΔ.Mi-WPRE post activation were 1.13 and 1.17. Activation of CAR-T cells with vector pCsin-NB.hEF1αΔ.Mi-WPRE did not lead to extra and inducible transcription, which indicated the stability of the optimized vectors and TENM promoter.

### Example 9: IL-2 Dependent Proliferation of CAR-T Cells Generated with the Lentiviral Vectors pCsin-hEF1α-WPRE and pCsin-NB.hEF1αΔ.Mi-WPRE

To investigate the influence of the optimized TENM promoter on proliferation, we tested the expansion performance of CAR-T cells generated with the lentiviral vectors pCsin-hEF1α-WPRE and pCsin-NB.hEF1αΔ.Mi-WPRE. In this example, the target gene is CAR #6 (2,652 bp). As described in Example 1, T cells in cryopreserved PBMC were enriched and sorted with magnetic beads. After sorting, the cells were incubated with activated beads and cultured in G-Rex plates. After transduction (Day 0), all groups were supplemented with complete medium with IL-2 for expansion, and the culture was continued with complete medium or IL-2 deprived medium from Day 6. Cell count and viability were also monitored from Day 6.

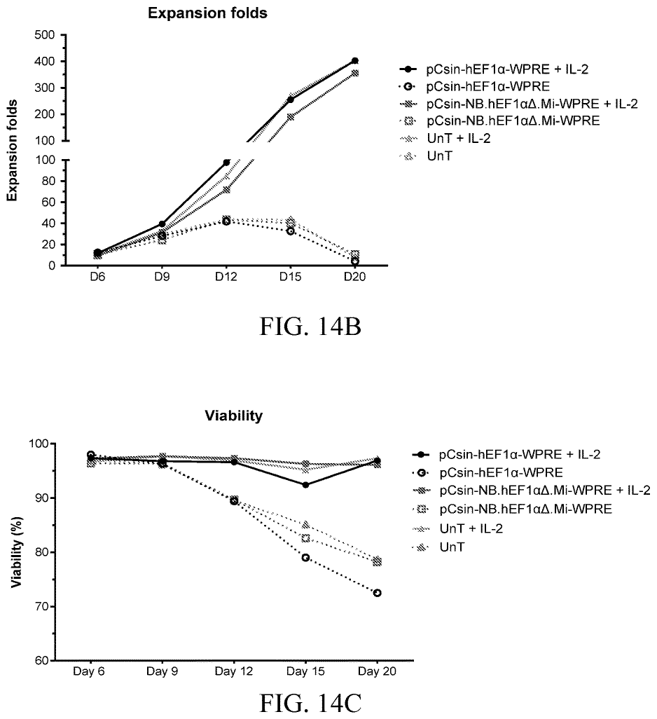

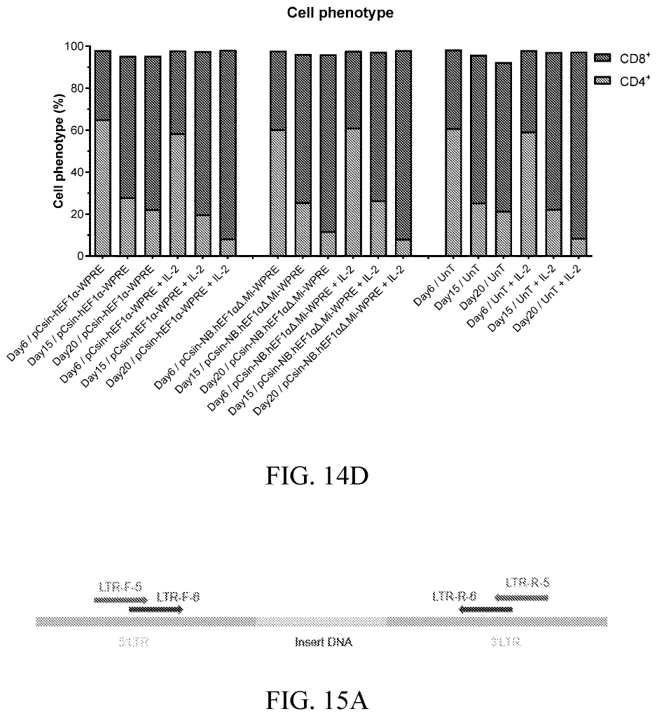

As shown in FIG. 14A, cell diameters of each group were 11.0, 11.0, 11.1, 11.1, 10.7 and 10.8 μm on Day 6, and showed significant difference from Day 12. At the conclusion of study, the diameter values of groups with IL-2 supplement were 10.1, 10.1 and 10.2 μm, while the diameter values of groups without IL-2 were 8.7, 8.7 and 8.8 μm. Similar with the above tendency, expansion folds showed dramatical difference from Day 12, and the expansion folds of groups with IL-2 supplement were 402.9, 355.4 and 402.4 while those of groups without IL-2 were 4.2, 10.8 and 8.1 (FIG. 14B). As shown in FIG. 14C, viability of each group were 97.3%, 98.0%, 97.3%, 96.4%, 96.7% and 97.1% on Day 6, and showed significant difference from Day 12. At the conclusion of study, viability values of groups with IL-2 supplement were 96.9%, 96.1% and 97.3%, while viability values of groups without IL-2 were 72.5%, 78.2%, and 78.7%. The results indicated cell viability was also highly dependent on IL-2 supplement. Similar variation tendency of cell phenotype were exhibited in FIG. 14D, which demonstrated the lentiviral vector and IL-2 did not alter T cell phenotype during the culture process. Groups with IL-2 deprivation showed shrink of cell diameter, decline of expansion as well viability. Cell activation, expansion and viability expressed closed correlation with IL-2 stimulation, and there was no prominent difference between lentiviral vectors pCsin-hEF1α-WPRE and pCsin-NB.hEF1αΔ.Mi-WPRE, suggesting that the lentiviral vector pCsin-NB.hEF1αΔ.Mi-WPRE will not give rise to uncontrolled growth of cells.

### Example 10: Integration Section Sequencing of Lentiviral Vectors pCsin-hEF1α-WPRE and pCsin-NB.hEF1αΔ.Mi-WPRE

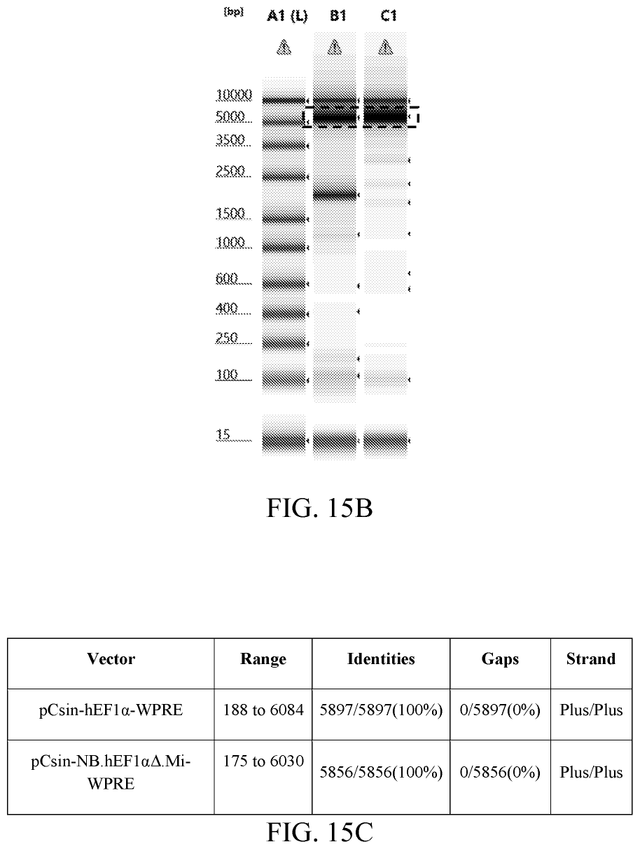

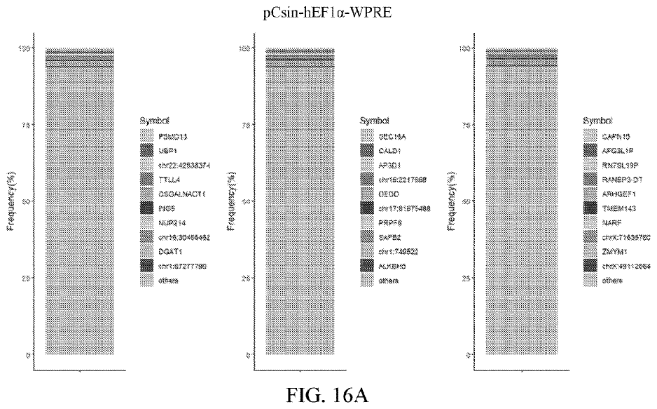

To investigate the transcription fidelity of the optimized TENM promoter, the integration sections in the target cell genome were amplificated and then sequenced. In this example, the target gene is CAR #6. Briefly, T cells were transduced using lentiviral vectors pCsin-hEF1α-WPRE and pCsin-NB.hEF1αΔ.Mi-WPRE, respectively. After DNA extraction from CAR-T cells, the target sequences were amplificated by nest PCR with high-fidelity Taq enzyme. As shown in FIG. 15A, the insertion fragment was amplificated with primer pairs LTR-F-5/LTR-R-5 and LTR-F-6/LTR-R-6 sequentially. The specific bands (marked in box, FIG. 15B) were located according to the product size and purified for further sequencing (A1 lane: DNA ladder; B1 lane: pCsin-hEF1α-WPRE; C1 lane: pCsin-NB.hEF1αΔ.Mi-WPRE). Sanger sequencing was then performed to acquire the accurate nucleotide data and the sequence alignment was performed using the online software Basic Local Alignment Search Tool (BLAST).

The sequence alignment results were shown in FIG. 15C. The insertion sequences were completely consistent with the sequences designed in the corresponding lentiviral vectors, indicating both lentiviral vectors pCsin-hEF1α-WPRE and pCsin-NB.hEF1αΔ.Mi-WPRE did not bring out any mutation event during the reverse transcription and integration process.

### Example 11: Insertion Location of Lentiviral Vectors pCsin-hEF1α-WPRE, pCsin-NB.hEF1αΔ.Mi and pCsin-NB.hEF1αΔ.Mi-WPRE

To investigate insertion sites of tendentious distribution or randomness, the gene loci of insertion fragments were detected by genome sequencing. In this example, the target gene is CAR #6. See PCT/CN2023/083705 for the detailed method. Briefly, the LTR containing sequences were enriched by amplicon-based method and then sequenced, the lentivirus integration sites could be determined by aligning these sequences to the human reference genome (GRCh38/hg38). Subsequently, the frequency and distribution of these integration sites were analyzed, including their distribution on tumorigenesis associated genes, to evaluate the safety of these integration sites.

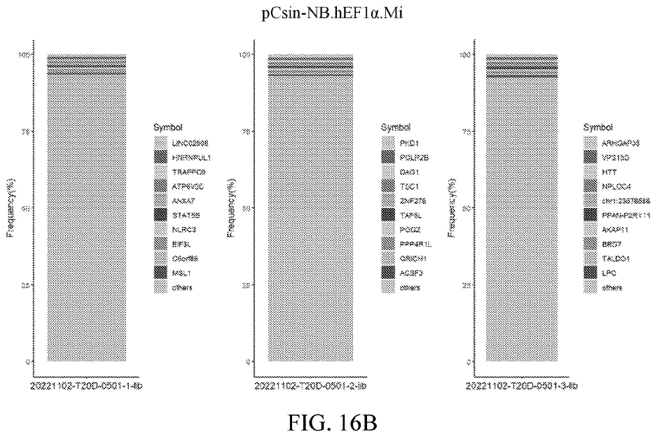

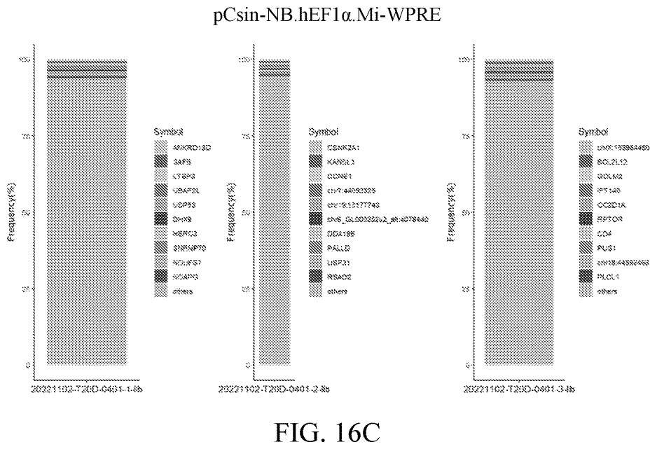

As shown in FIG. 16A-16C, the integration loci of top ten proportion in three libraries of lentiviral vectors pCsin-hEF1α-WPRE, pCsin-NB.hEF1αΔ.Mi and pCsin-NB.hEF1αΔ.Mi-WPRE were totally different, suggesting that the integration sites of the lentiviral vectors pCsin-hEF1α-WPRE, pCsin-NB.hEF1αΔ.Mi and pCsin-NB.hEF1αΔ.Mi-WPRE were completely random and there was no high-frequency integration event. For the lentiviral vector pCsin-hEF1α-WPRE, 13,199 integration events and 3,051 integration sites were detected in total, and the mean frequency was 1/3051=0.03%. For the optimized lentiviral vector pCsin-NB.hEF1αΔ.Mi, 11,255 integration events and 2,739 integration sites were detected in total, and the mean frequency was 1/2,739=0.04%. For the optimized lentiviral vector pCsin-NB.hEF1αΔ.Mi-WPRE, 12,177 integration events and 2935 integration sites were detected in total, and the mean frequency was 1/2,935=0.03%.

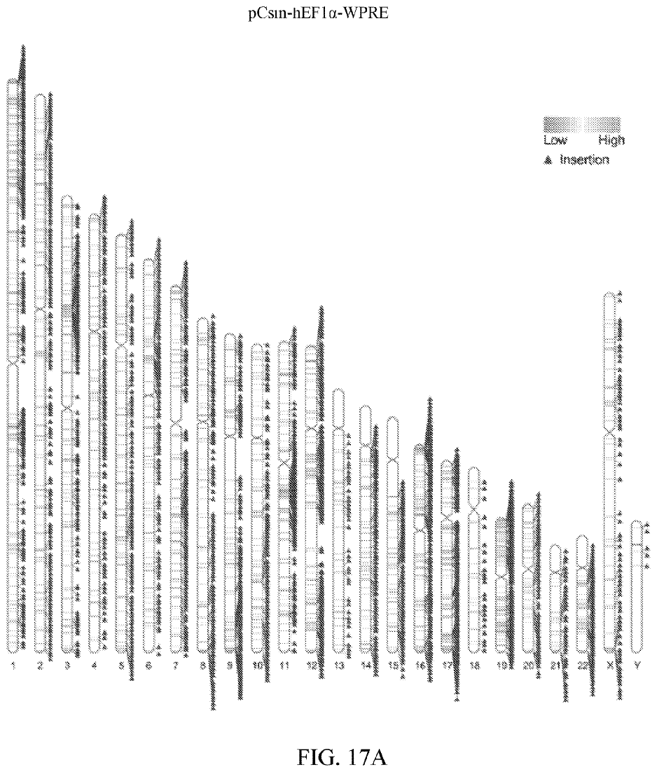

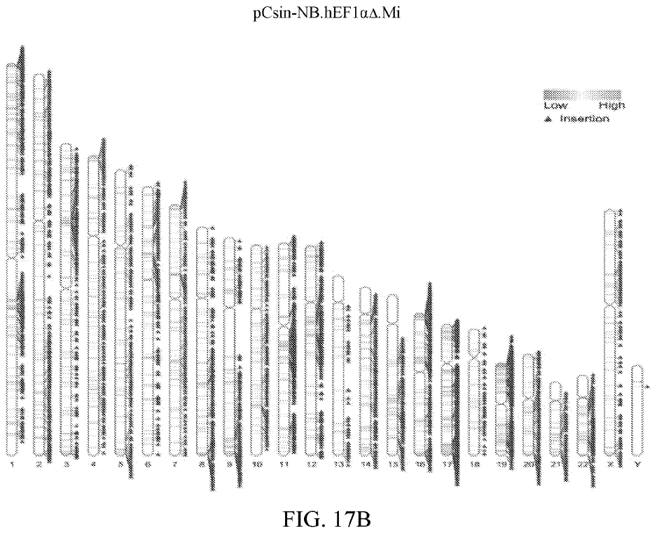

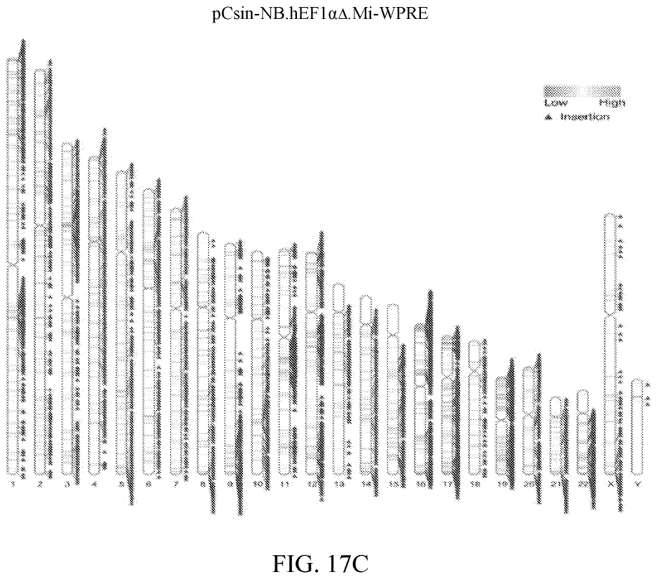

Results of FIGS. 17A-17C indicated the random distribution of insertion sites on chromosomes, which further showed the random insertion property of the optimized lentiviral vector pCsin-hEF1α-WPRE, pCsin-NB.hEF1αΔ.Mi and pCsin-NB.hEF1αΔ.Mi-WPRE. According to the statistics of genome annotation files, the proportion of exon, intron and intergenic regions in human GRCh38 genome is about 0.04:0.46:0.50. In this example, intergenic region accounting for 50% only contained about 10% integration sites, while the region where 50% of genes were located (exon and intron) contained about 90% integration sites, so the distribution of integration sites tended to be integrated into the region where genes were located.

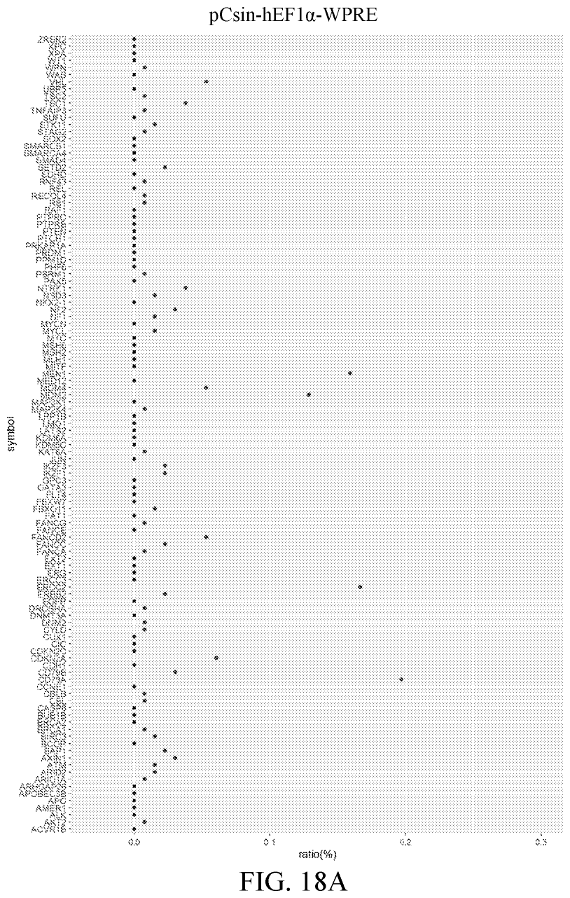

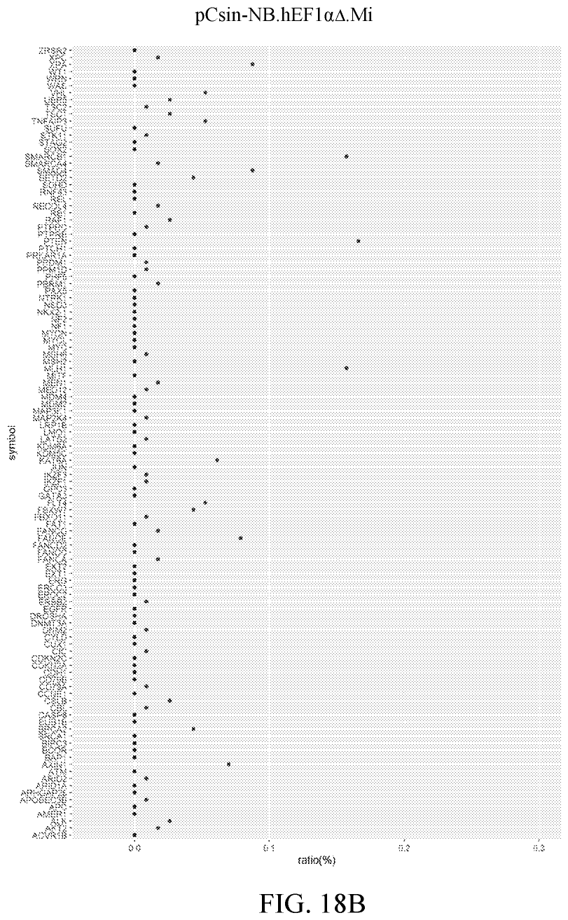

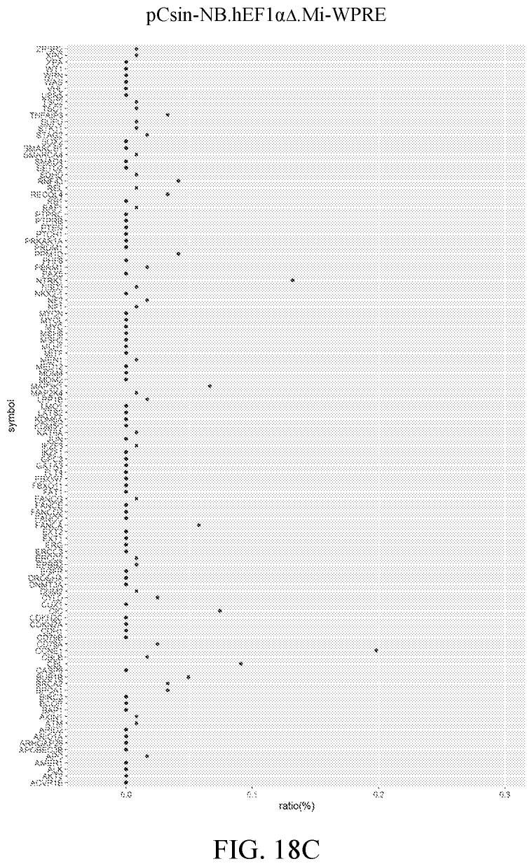

We paid extra attention to the integration events within oncogenes (100 kb upstream and 100 kb downstream). As shown in FIGS. 18A-18C, the insertion frequencies within oncogenes (GRCh38 COSMIC v96, updated by Aug. 11, 2022) were marked. For lentiviral vectors pCsin-hEF1α-WPRE, pCsin-NB.hEF1αΔ.Mi and pCsin-NB.hEF1αΔ.Mi-WPRE, the maximum percentage approached 0.2%. For lentiviral vectors pCsin-hEF1α-WPRE, pCsin-NB.hEF1αΔ.Mi, pCsin-NB.hEF1αΔ.Mi-WPRE insertion events with a frequency less than 0.1% within oncogenes were 42, 44 and 41 respectively, while insertion events with frequencies between 0.1% and 0.2% within oncogenes were 4, 3 and 2 respectively. The results showed that both the optimized lentiviral vectors pCsin-NB.hEF1αΔ.Mi and pCsin-NB.hEF1αΔ.Mi-WPRE did not show a higher risk on carcinogenicity than the classical lentiviral vector pCsin-hEF1α-WPRE.

### Example 12: Construction of Lentiviral Vector pCsin-NB.hEF1αΔ.Mi-g-WPRE

To eliminate the potential risk of the undesired generation of open reading frames (ORF), we mutated initiation codon sequences in MVM introns and generated MVM introns Mi-g (SEQ ID NO: 13). Then optimized lentiviral vector pCsin-NB.hEF1αΔ.Mi-g-WPRE was constructed with Mi-g sequence (FIG. 19).

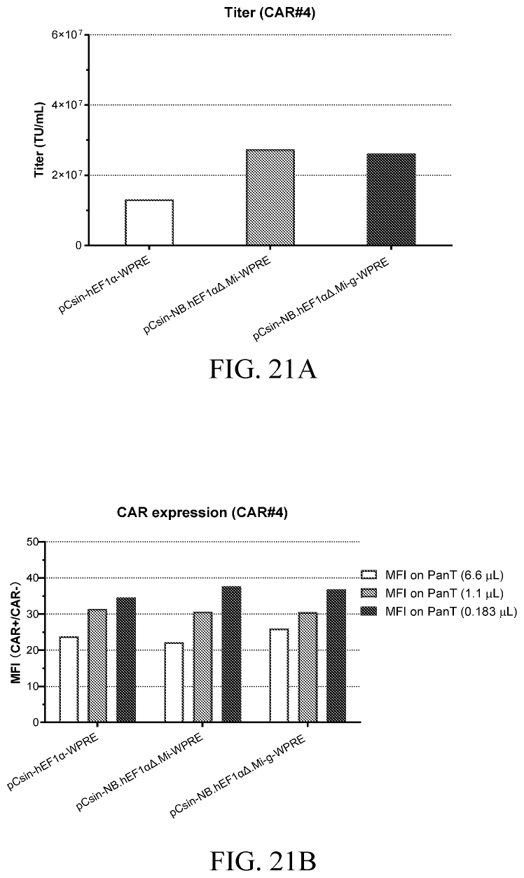

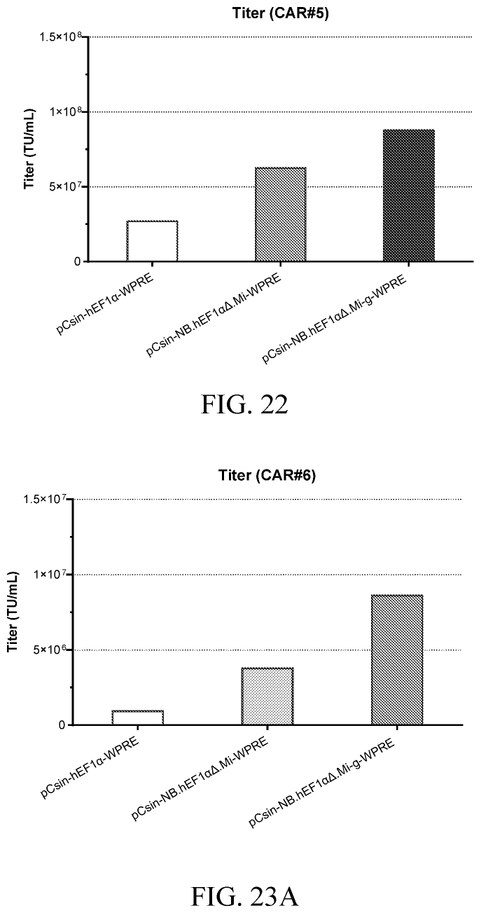

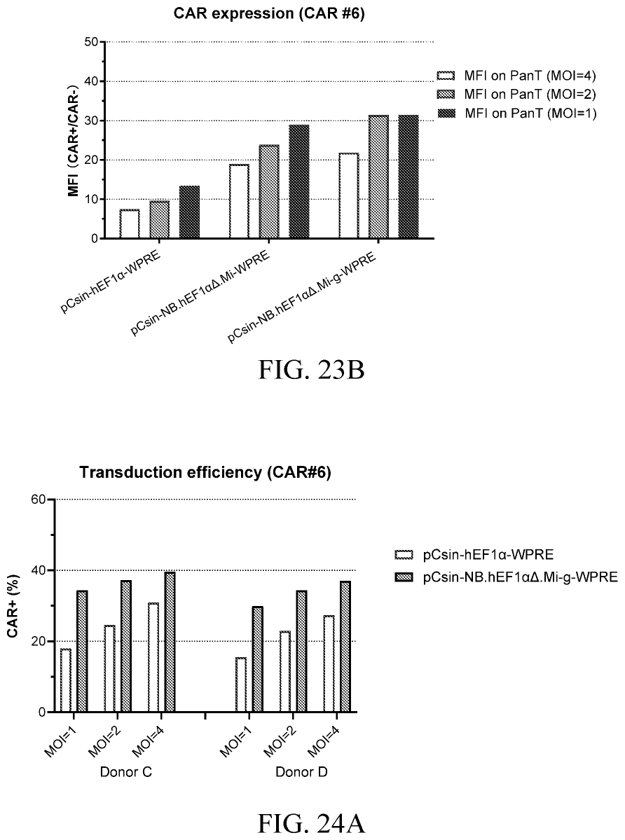

In this example, the target genes include CAR #3, CAR #4 (1,872 bp), CAR #5 (1,464 bp) and CAR #6. The methods in Example 1 were used for lentivirus packaging, positive cell detection and expression level determination. The supernatant containing lentivirus was harvested for titer determination. As shown in FIG. 20A, FIG. 21A, FIG. 22, and FIG. 23A, titers of lentiviral vector pCsin-hEF1α-WPRE were 6.76×10⁶, 1.31×10⁷, 2.75×10⁷ and 9.85×10⁵ Tu/ml, titers of lentiviral vector pCsin-NB.hEF1αΔ.Mi-WPRE were 2.77×10⁷, 2.73×10⁷, 6.30×10⁷ and 3.82×10⁶ Tu/ml, and titers of lentiviral vector pCsin-NB.hEF1αΔ.Mi-g-WPRE were 2.48×10⁷, 2.62×10⁷, 8.83×10⁷ and 8.66×10⁶ Tu/ml respectively. Compared with lentiviral vector pCsin-hEF1α-WPRE, pCsin-NB.hEF1αΔ.Mi-WPRE showed significantly better virus titers, which promoted 4.10, 2.08, 2.29 and 3.88 folds respectively. Lentiviral vector pCsin-NB.hEF1αΔ.Mi-g-WPRE also showed significantly better virus titers than pCsin-hEF1α-WPRE vector, which promoted 3.67, 2.00, 3.21 and 8.99 folds respectively. Compared with the classical lentiviral vector pCsin-hEF1α-WPRE, lentiviral vector pCsin-NB.hEF1αΔ.Mi-WPRE significantly improved the titer performance, which was consistent with the results shown in Example 5. The lentiviral vector pCsin-NB.hEF1αΔ.Mi-g-WPRE exhibited equivalent or even better titer promotion than lentiviral vector pCsin-NB.hEF1αΔ.Mi-WPRE, especially for the target gene with low absolute titer, such as CAR #6. Meanwhile, as shown in FIG. 20B, FIG. 21B and FIG. 23B, lentiviral vectors pCsin-NB.hEF1αΔ.Mi-WPRE and pCsin-NB.hEF1αΔ.Mi-g-WPRE showed higher expression level of target genes than that of pCsin-hEF1α-WPRE vector.

### Example 13: Transduction Efficiency of Lentiviral Vectors pCsin-hEF1α-WPRE and pCsin-NB.hEF1αΔ.Mi-g-WPRE

To determine the transduction efficiency of the optimized lentiviral vectors, primary T cells derived from two donors were infected by the lentivirus packaged using lentiviral vectors pCsin-hEF1α-WPRE and pCsin-NB.hEF1αΔ.Mi-g-WPRE. In this example, the methods for lentivirus packaging and titer detection in Example 1 were used, and the target gene was CAR #6. The cryopreserved PBMC was sorted using CD4 and CD8 antibody conjugated magnetic beads, then the sorted cells were incubated with activated beads. After transduction at MOI=1, 2, and 4, T cells were analyzed by flow cytometry to determine the proportion of CAR+ cells, and the Median Fluorescence Intensity (MFI) of positive cells was detected to calculate CAR density.

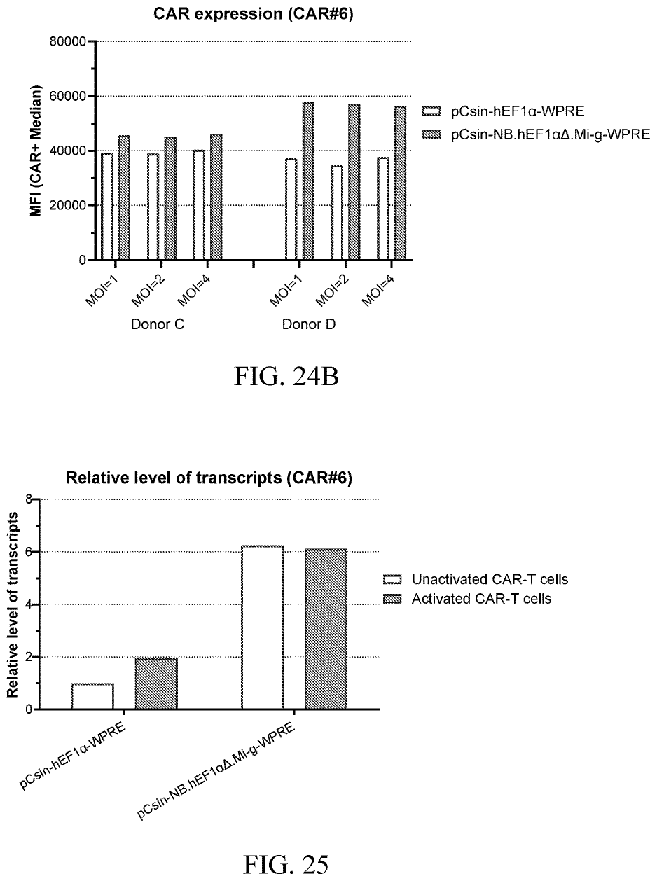

As shown in FIG. 24A, in the condition of MOI=1, 2, or 4, the percentages of CAR+ cells (Donor C) with pCsin-hEF1α-WPRE were 18.00%, 24.60% and 30.90% respectively. Percentages of pCsin-NB.hEF1αΔ.Mi-g-WPRE were 34.40%, 37.26% and 39.64%. Parallelly, the percentages of CAR+ cells (Donor D) with pCsin-hEF1α-WPRE were 15.53%, 22.90% and 27.34% respectively. Percentages of pCsin-NB.hEF1αΔ.Mi-g-WPRE were 29.91%, 34.29% and 37.02%, suggesting higher transduction efficiency of lentiviral vector pCsin-NB.hEF1αΔ.Mi-g-WPRE. Meanwhile, CAR expression level of lentiviral vector pCsin-NB.hEF1αΔ.Mi-g-WPRE was higher that of pCsin-hEF1α-WPRE (FIG. 24B). The results indicate that the optimized lentiviral vector could promote both transduction efficiency and target gene expression on primary T cells.

### Example 14: Transcription Level Post-Activation of Lentiviral Vectors pCsin-hEF1α-WPRE and pCsin-NB.hEF1αΔ.Mi-g-WPRE

To determine whether the T cell activation will promote transcription level, we compared the transcript levels of lentiviral vectors pCsin-hEF1α-WPRE and pCsin-NB.hEF1αΔ.Mi-g-WPRE. In this example, the target gene is CAR #6.

CAR-T cells were prepared as description in Example 1. LS-1034 tumor cells were cultured in RPMI 1640 medium containing 10% FBS (fetal bovine serum). The methods in Example 8 were used for CAR-T cell activation and transcript measurement. As shown in FIG. 25, relative transcription levels of lentiviral vectors pCsin-hEF1α-WPRE and pCsin-NB.hEF1αΔ.Mi-g-WPRE without activation were 1.00 and 6.25, while those were 1.96 and 6.12 post activation. For CAR #6, the transcript level of lentiviral vector pCsin-NB.hEF1αΔ.Mi-g-WPRE was much higher than that of pCsin-hEF1α-WPRE, which was consistent with MFI result in FIG. 23B. However, lentiviral vector pCsin-NB.hEF1αΔ.Mi-g-WPRE did not lead to inducible transcription post activation, which indicated the stability of the optimized vector and TENM promoter. Surprisingly, the classical lentiviral vector pCsin-hEF1α-WPRE showed elevated transcription post co-incubation, which suggested the potential inducible characteristic.

### Example 15: Transcription Termination of Lentiviral Promoters hEF1α, NB.hEF1αΔ.Mi and NB.hEF1αΔ.Mi-g

To evaluate the transcription termination ability of the optimized TENM promoter, the vectors were constructed and shown in FIG. 26. In this example, eGFP (enhanced green fluorescent protein) element was used to determine the termination capacity of promoters hEF1α, NB.hEF1αΔ.Mi and NB.hEF1αΔ.Mi-g.

HEK293T cells were inoculated into T75 flasks at a proper cell density, and cultured at 37 °C for 1 day. Plasmids in FIG. 26 were diluted in 250 μL of DMEM medium and mixed with PEIpro-HQ at a plasmid:PEI=1:1.5 ratio. The mixture was inoculated at room temperature for 10 min before added to T75 flask. Fluorescence was observed and photographed through a fluorescence microscope at 48 h post transfection. Then, trypsin digestion and centrifugation were performed to collect cells for flow cytometry from which GFP positive rate and expression level data were obtained.

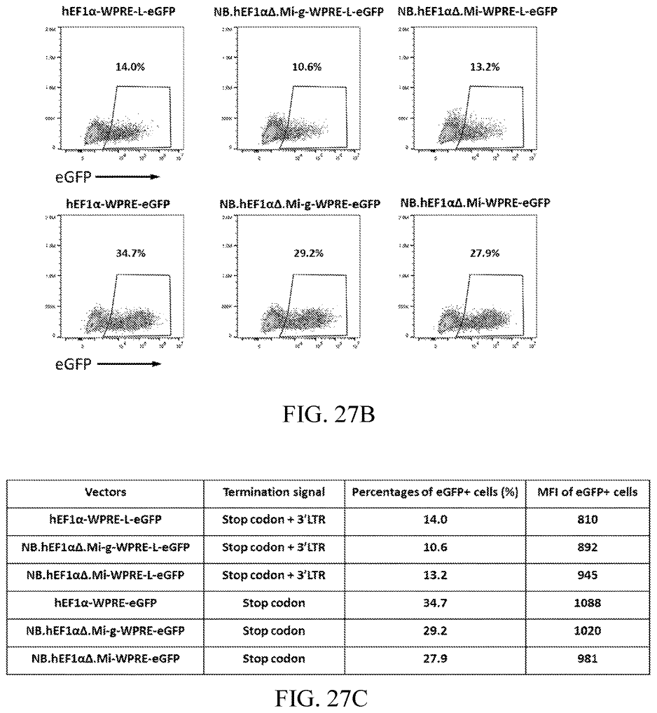

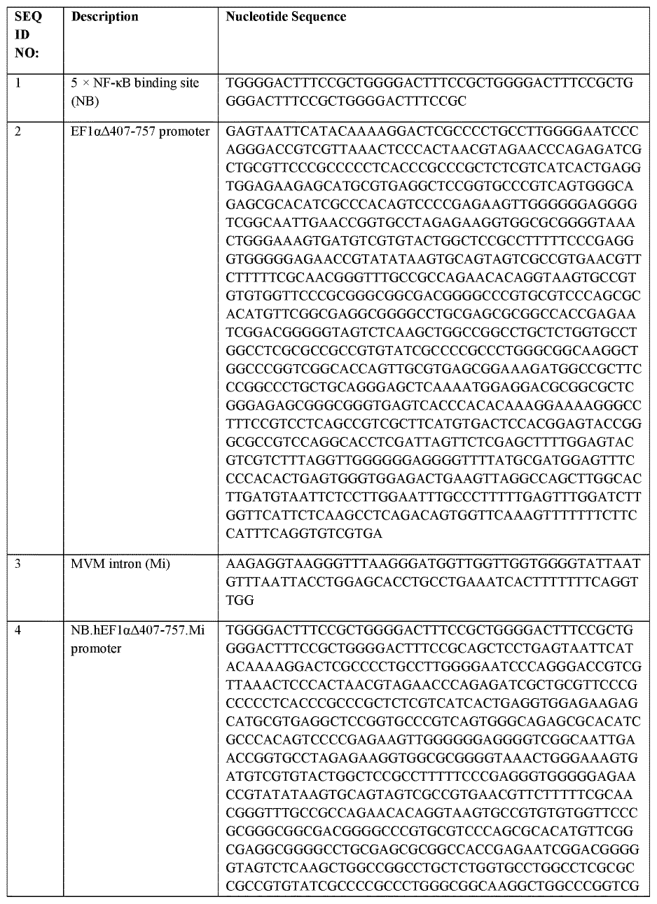

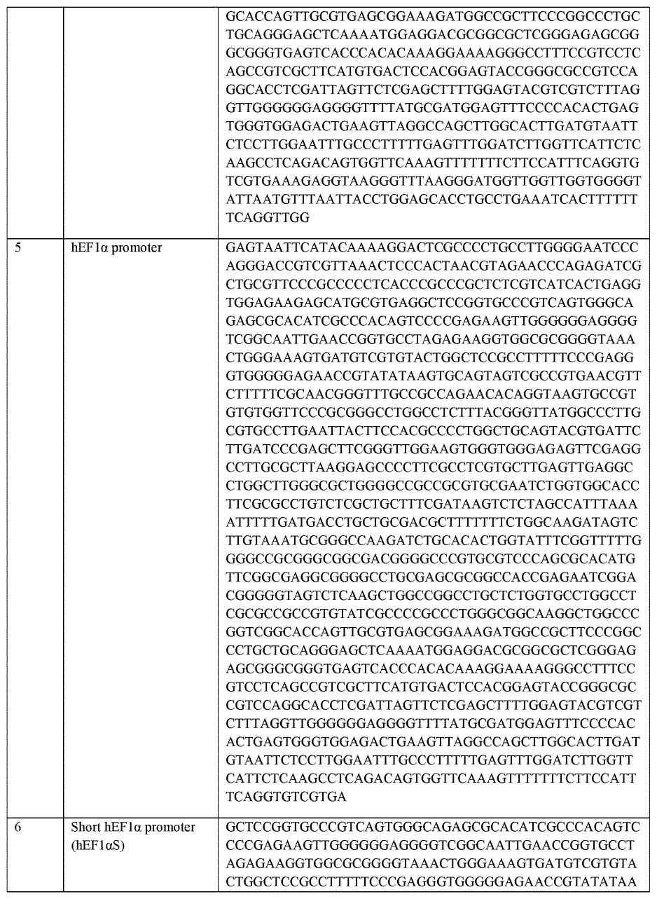

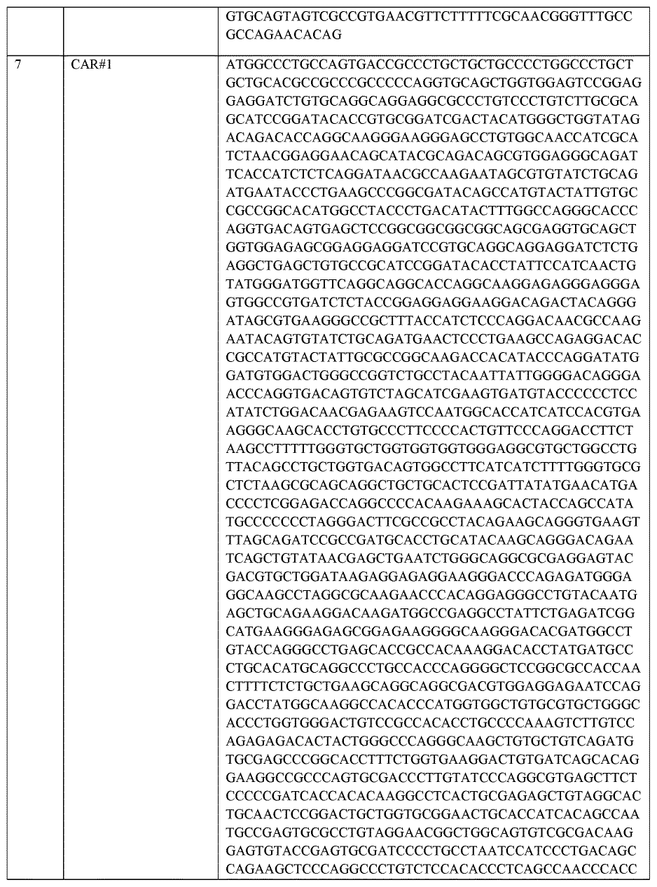

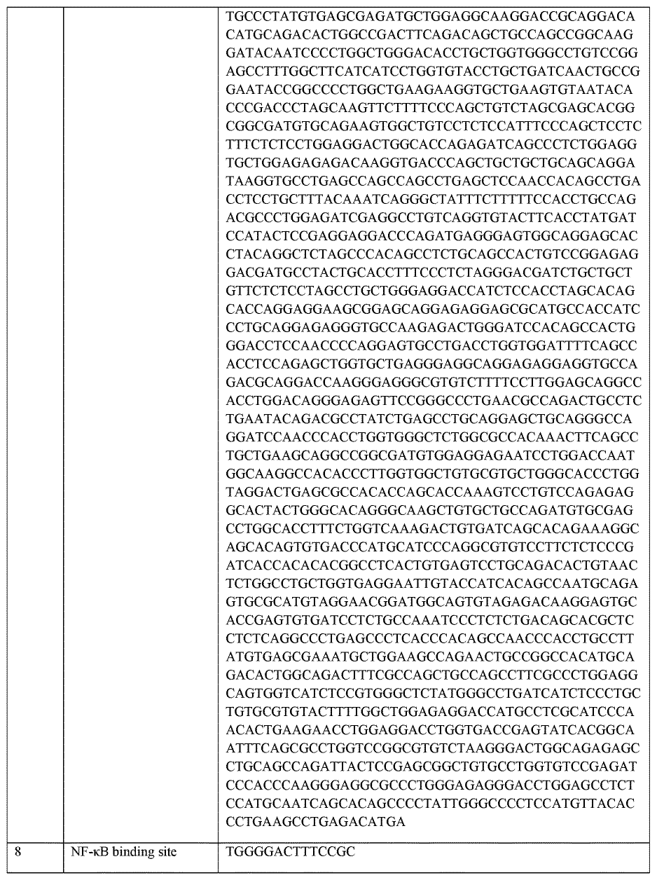

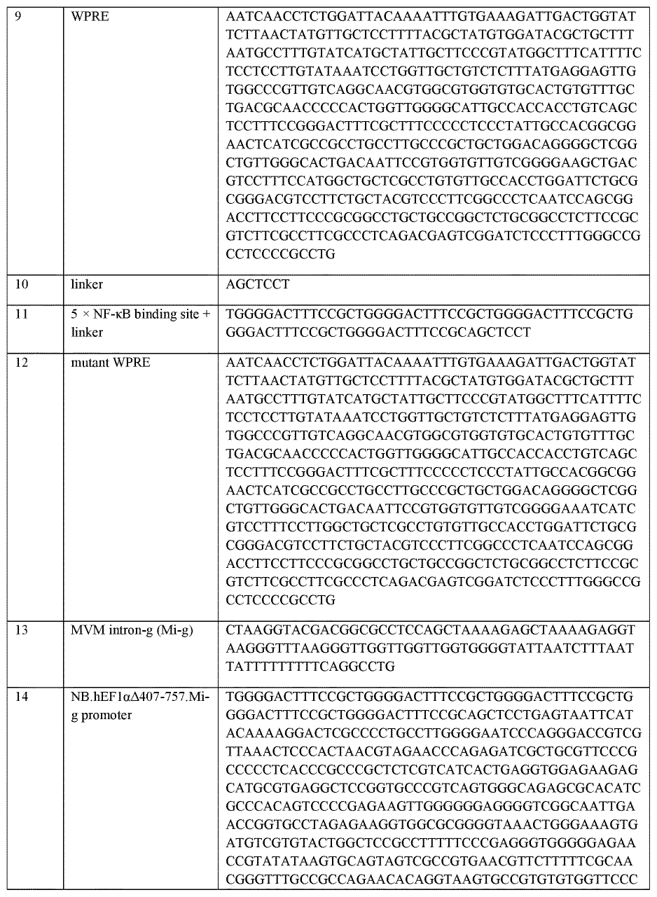

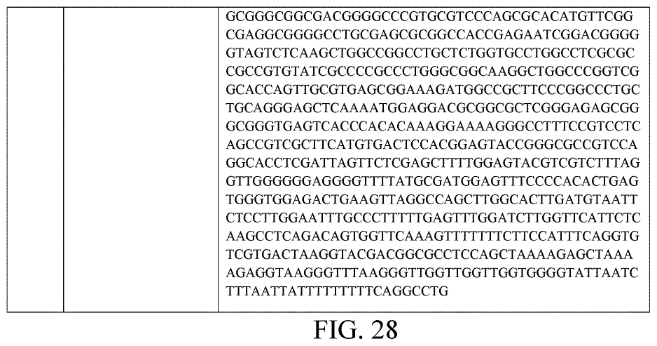

As shown in FIG. 27A, indicated by eGFP expression, stop codon could effectively terminate transcription of hEF1α, NB.hEF1αΔ.Mi and NB.hEF1αΔ.Mi-g promoters in most cells. Neither percentages nor MFI of eGFP+ cells showed difference in transcriptional termination ability, and 3′LTR sequence could provide extra termination signal (FIGS. 27B-27C). The TENM promoters (e.g. NB.hEF1αΔ.Mi and NB.hEF1αΔ.Mi-g promoters) might even display slightly better transcription termination ability than that of hEF1α promoter.

In summary, we constructed a chimeric TENM promoter containing a double intron and a transcription factor binding sequence, and applied it to a lentiviral vector, maintaining or enhancing the expression level of the target gene, and enhancing the titer of lentivirus, especially for the target gene with large size. One of the preferred lentiviral vectors pCsin-NB.hEF1αΔ.Mi-g-WPRE can additionally increase transduction efficiency and CAR density. Both in vitro and in vivo efficacy study indicate the robust tumor killing ability of the optimized lentiviral vectors, which is equivalent to the performance of the traditional lentiviral vector. The TENM promoter does not displayed inducible transcription, in the condition of target cell activation. Simultaneously, poly A sequences in 3′LTR region can efficiently terminate transcription of the TENM promoter, which avoid the undesired impact to the downstream sequences. At last, there is no evidence showing that the optimized lentiviral vectors have potential risk of tendentious integration and oncogenicity. In conclusion, the TENM promoters and the optimized lentiviral vectors can effectively promote the lentivirus packaging and delivering efficiency, without undesired performance loss and risk of safety.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A regulatory sequence, comprising:
(a) an EF1α promoter;
(b) a small intron region of the minute virus of mice (MVM); and
(c) a NF-κB binding site.

2. The regulatory sequence of claim 1, wherein the EF1α promoter is a truncated EF1α promoter.

3. The regulatory sequence of claim 1 or claim 2, wherein the EF1α promoter does not contain any intron sequence.

4. The regulatory sequence of claim 1 or claim 2, wherein the EF1α promoter does not contain nucleotides 407-757 of the full length EF1α promoter.

5. The regulatory sequence of any one of claims 1-4, wherein the EF1α promoter comprises a sequence that is at least 70%, 80%, 85%, 90% or 100% identical to SEQ ID NO: 2, 5 or 6.

6. The regulatory sequence of any one of claims 1-5, wherein the small intron region of MVM comprises a sequence that is at least 70%, 80%, 85%, 90% or 100% identical to SEQ ID NO: 3 or 13.

7. The regulatory sequence of any one of claims 1-6, wherein the NF-κB binding site comprises a NF-κB binding sequence that is at least 70%, 80%, 85%, 90% or 100% identical to SEQ ID NO: 8.

8. The regulatory sequence of any one of claims 1-7, wherein the regulatory sequence comprises a sequence that is at least 70%, 80%, 85%, 90% or 100% identical to SEQ ID NO: 4 or 14.

9. The regulatory sequence of any one of claims 1-8, wherein the regulatory sequence further comprises a WPRE.

10. The regulatory sequence of any one of claims 1-8, wherein the regulatory sequence does not comprise a WPRE.

11. The regulatory sequence of any one of claims 1-10, wherein the NF-κB binding site comprises at least three copies of a NF-κB binding sequence (e.g., SEQ ID NO: 8).

12. The regulatory sequence of any one of claims 1-11, wherein the NF-κB binding site comprises about 4 to 6 copies of a NF-κB binding sequence (e.g., SEQ ID NO: 8).

13. The regulatory sequence of any one of claims 1-12, wherein the NF-κB binding site comprises 5 copies of a NF-κB binding sequence (e.g., SEQ ID NO: 8), and the NF-κB binding site is at least 70%, 80%, 85%, 90% or 100% identical to SEQ ID NO: 1.

14. The regulatory sequence of any one of claims 1-13, wherein the NF-κB binding site further comprises a linker sequence (e.g., SEQ ID NO: 10), and the NF-κB binding site is at least 70%, 80%, 85%, 90% or 100% identical to SEQ ID NO: 11.

15. The regulatory sequence of any one of claims 1-14, wherein the total length of the regulatory sequence is less than 1,200 bp.

16. The regulatory sequence of any one of claims 1-15, wherein the regulatory sequence comprises, from the 5′ end to the 3′ end: (a) the NF-κB binding site; (b) the EF1α promoter; and (c) the small intron region of MVM.

17. The regulatory sequence of any one of claims 1-15, wherein the regulatory sequence comprises, from the 5′ end to the 3′ end: (a) the small intron region of MVM; (b) the NF-κB binding site; and (c) the EF1α promoter.

18. The regulatory sequence of any one of claims 1-15, wherein the regulatory sequence comprises, from the 5′ end to the 3′ end: (a) the NF-κB binding site; (b) the small intron region of MVM; and (c) the EF1α promoter.

19. A vector, comprising: the regulatory sequence of any one of claims 1-18 and a gene of interest (GOI).

20. The vector of claim 19, wherein the GOI encodes a chimeric antigen receptor (CAR), a modified T-cell receptor (TCR), an antibody, a fusion protein, an enzyme, a soluble protein, a membrane protein, a structural protein, a ribosome protein, a zymogen, a cell surface receptor protein, a transcriptional regulatory protein, a translational regulatory protein, a chromatin protein, a hormone, a cell cycle regulatory protein, a G protein, a neuroactive peptide, an immunomodulatory protein, a blood component protein, an ion gate protein, a heat shock protein, a dihydrofolate reductase, an antibiotic resistance protein, a functional fragment of any one of the proteins, and any combination thereof.

21. The vector of any one of claims 19 and 20, wherein the vector is a lentivirus vector.

22. The vector of any one of claims 19-21, wherein the vector further comprises a 5′ long terminal repeat (LTR) and/or a 3′ LTR.

23. The vector of any one of claims 19-22, wherein the vector further comprises a Psi (Ψ) packaging signal.

24. The vector of any one of claims 19-23, wherein the vector further comprises a heterologous polyadenylation sequence.

25. The vector of any one of claims 19-24, wherein the vector further comprises a cytomegalovirus (CMV) promoter.

26. The vector of any one of claims 19-25, wherein the vector comprises, from the 5′ end to the 3′ end: (a) the CMV promoter, (b) the 5′ LTR, (c) the regulatory sequence, (d) the GOI, and (e) the 3′ LTR.

27. The vector of any one of claims 19-26, wherein the vector further comprises a Rev-responsive element (RRE).

28. The vector of any one of claims 19-27, wherein the vector further comprises a central polypurine tract (cPPT).

29. A packaging cell comprising the vector of any one of claims 19-28.

30. A virus particle produced by the packaging cell of claim 29.

31. A virus particle comprising a nucleic acid comprising the regulatory sequence of any one of claims 1-18.

32. The virus particle of claim 31, wherein the virus particle is a lentivirus particle.

33. The virus particle of claim 31 or 32, wherein the nucleic acid is RNA.

34. A method of expressing a protein of interest, comprising:
(a) producing virus particles using the packaging cell of claim 29;
(b) infecting a host cell with the virus particles; and
(c) expressing a protein of interest using the host cell.

35. The method of claim 34, wherein the method can increase the expression level of the protein of interest by about or at least 10%, 20%, 30%, 40%, 50%, 60%, 70%, 80%, 90%, 100%, 120%, 140%, 160%, 180%, 200%, 300%, 400%, 500%, 600%, or 700%, as compared to a control sequence that is different from the TENM sequence.

36. The method of claim 34 or claim 35, wherein the method can increase the titer of the virus particles by about or at least 10%, 20%, 30%, 40%, 50%, 60%, 70%, 80%, 90%, 100%, 120%, 140%, 160%, 180%, 200%, 300%, 400%, 500%, 600%, or 700%, as compared to a control sequence that is different from the TENM sequence.

37. A method of making an immune cell, comprising
(a) isolating immune cells from a subject; and
(b) infecting the isolated immune cells using the virus particles of any one of claims 30-33.

38. An immune cell comprising the regulatory sequence of any one of claims 1-18.

39. The immune cell of claim 38, wherein the immune cell is selected from the group consisting of a cytotoxic T cell, a helper T cell, a natural killer (NK) cell, an iNK-T cell, an NK-T like cell, an αβT cell and a γδT cell.

40. The immune cell of claim 38 or claim 39, wherein the immune cell comprises a gene of interest that is operably linked to the regulatory sequence of any one of claims 1-18, wherein the gene of interest encodes a chimeric antigen receptor (CAR) or modified T-cell receptor (TCR).

41. The immune cell of claim 39, wherein the immune cell expresses the chimeric antigen receptor (CAR) or modified T-cell receptor (TCR).

42. A method of treating a subject having cancer, the method comprising administering a therapeutically effective amount of a composition comprising the virus particle of claims 30-33, or the immune cell of claims 38-41, to the subject.

43. The method of claim 42, wherein the cancer is solid tumor.

44. The method of claim 42, wherein the cancer is hematologic cancer.

45. A pharmaceutical composition comprising the virus particle of claims 30-33 or the immune cell of claims 38-41, and a pharmaceutically acceptable carrier.


SEQUENCE LISTING (14 sequences)

SEQ ID NO: 1 - DNA, 70 bp, synthetic construct
  source 1..70: mol_type = other DNA
tggggactttccgctggggactttccgctggggactttccgctggggactttccgctggggactttccgc

SEQ ID NO: 2 - DNA, 984 bp, synthetic construct
  source 1..984: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 3 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 4 - DNA, 1153 bp, synthetic construct
  source 1..1153: mol_type = other DNA
tggggactttccgctggggactttccgctggggactttccgctggggactttccgctggggactttccgcagctcctgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaagaggtaagggtttaagggatggttggttggtggggtattaatgtttaattacctggagcacctgcctgaaatcactttttttcaggttgg

SEQ ID NO: 5 - DNA, 1335 bp, synthetic construct
  source 1..1335: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 6 - DNA, 230 bp, synthetic construct
  source 1..230: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 7 - DNA, 3906 bp, synthetic construct
  source 1..3906: mol_type = other DNA
atggccctgccagtgaccgccctgctgctgcccctggccctgctgctgcacgccgcccgcccccaggtgcagctggtggagtccggaggaggatctgtgcaggcaggaggcgccctgtccctgtcttgcgcagcatccggatacaccgtgcggatcgactacatgggctggtatagacagacaccaggcaagggaagggagcctgtggcaaccatcgcatctaacggaggaacagcatacgcagacagcgtggagggcagattcaccatctctcaggataacgccaagaatagcgtgtatctgcagatgaataccctgaagcccggcgatacagccatgtactattgtgccgccggcacatggcctaccctgacatactttggccagggcacccaggtgacagtgagctccggcggcggcggcagcgaggtgcagctggtggagagcggaggaggatccgtgcaggcaggaggatctctgaggctgagctgtgccgcatccggatacacctattccatcaactgtatgggatggttcaggcaggcaccaggcaaggagagggagggagtggccgtgatctctaccggaggaggaaggacagactacagggatagcgtgaagggccgctttaccatctcccaggacaacgccaagaatacagtgtatctgcagatgaactccctgaagccagaggacaccgccatgtactattgcgccggcaagaccacatacccaggatatggatgtggactgggccggtctgcctacaattattggggacagggaacccaggtgacagtgtctagcatcgaagtgatgtacccccctccatatctggacaacgagaagtccaatggcaccatcatccacgtgaagggcaagcacctgtgcccttccccactgttcccaggaccttctaagcctttttgggtgctggtggtggtgggaggcgtgctggcctgttacagcctgctggtgacagtggccttcatcatcttttgggtgcgctctaagcgcagcaggctgctgcactccgattatatgaacatgacccctcggagaccaggccccacaagaaagcactaccagccatatgccccccctagggacttcgccgcctacagaagcagggtgaagtttagcagatccgccgatgcacctgcatacaagcagggacagaatcagctgtataacgagctgaatctgggcaggcgcgaggagtacgacgtgctggataagaggagaggaagggacccagagatgggaggcaagcctaggcgcaagaacccacaggagggcctgtacaatgagctgcagaaggacaagatggccgaggcctattctgagatcggcatgaagggagagcggagaaggggcaagggacacgatggcctgtaccagggcctgagcaccgccacaaaggacacctatgatgccctgcacatgcaggccctgccacccaggggctccggcgccaccaacttttctctgctgaagcaggcaggcgacgtggaggagaatccaggacctatggcaaggccacacccatggtggctgtgcgtgctgggcaccctggtgggactgtccgccacacctgccccaaagtcttgtccagagagacactactgggcccagggcaagctgtgctgtcagatgtgcgagcccggcacctttctggtgaaggactgtgatcagcacaggaaggccgcccagtgcgacccttgtatcccaggcgtgagcttctcccccgatcaccacacaaggcctcactgcgagagctgtaggcactgcaactccggactgctggtgcggaactgcaccatcacagccaatgccgagtgcgcctgtaggaacggctggcagtgtcgcgacaaggagtgtaccgagtgcgatcccctgcctaatccatccctgacagccagaagctcccaggccctgtctccacaccctcagccaacccacctgccctatgtgagcgagatgctggaggcaaggaccgcaggacacatgcagacactggccgacttcagacagctgccagccggcaaggatacaatcccctggctgggacacctgctggtgggcctgtccggagcctttggcttcatcatcctggtgtacctgctgatcaactgccggaataccggcccctggctgaagaaggtgctgaagtgtaatacacccgaccctagcaagttcttttcccagctgtctagcgagcacggcggcgatgtgcagaagtggctgtcctctccatttcccagctcctctttctctcctggaggactggcaccagagatcagccctctggaggtgctggagagagacaaggtgacccagctgctgctgcagcaggataaggtgcctgagccagccagcctgagctccaaccacagcctgacctcctgctttacaaatcagggctatttctttttccacctgccagacgccctggagatcgaggcctgtcaggtgtacttcacctatgatccatactccgaggaggacccagatgagggagtggcaggagcacctacaggctctagcccacagcctctgcagccactgtccggagaggacgatgcctactgcacctttccctctagggacgatctgctgctgttctctcctagcctgctgggaggaccatctccacctagcacagcaccaggaggaagcggagcaggagaggagcgcatgccaccatccctgcaggagagggtgccaagagactgggatccacagccactgggacctccaaccccaggagtgcctgacctggtggattttcagccacctccagagctggtgctgagggaggcaggagaggaggtgccagacgcaggaccaagggagggcgtgtcttttccttggagcaggccacctggacagggagagttccgggccctgaacgccagactgcctctgaatacagacgcctatctgagcctgcaggagctgcagggccaggatccaacccacctggtgggctctggcgccacaaacttcagcctgctgaagcaggccggcgatgtggaggagaatcctggaccaatggcaaggccacacccttggtggctgtgcgtgctgggcaccctggtaggactgagcgccacaccagcaccaaagtcctgtccagagaggcactactgggcacagggcaagctgtgctgccagatgtgcgagcctggcacctttctggtcaaagactgtgatcagcacagaaaggcagcacagtgtgacccatgcatcccaggcgtgtccttctctcccgatcaccacacacggcctcactgtgagtcctgcagacactgtaactctggcctgctggtgaggaattgtaccatcacagccaatgcagagtgcgcatgtaggaacggatggcagtgtagagacaaggagtgcaccgagtgtgatcctctgccaaatccctctctgacagcacgctcctctcaggccctgagccctcacccacagccaacccacctgccttatgtgagcgaaatgctggaagccagaactgccggccacatgcagacactggcagactttcgccagctgccagccttcgccctggaggcagtggtcatctccgtgggctctatgggcctgatcatctccctgctgtgcgtgtacttttggctggagaggaccatgcctcgcatcccaacactgaagaacctggaggacctggtgaccgagtatcacggcaatttcagcgcctggtccggcgtgtctaagggactggcagagagcctgcagccagattactccgagcggctgtgcctggtgtccgagatcccacccaagggaggcgccctgggagagggacctggagcctctccatgcaatcagcacagcccctattgggcccctccatgttacaccctgaagcctgagacatga

SEQ ID NO: 8 - DNA, 14 bp, synthetic construct
  source 1..14: mol_type = other DNA
tggggactttccgc

SEQ ID NO: 9 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 10 - skipped

SEQ ID NO: 11 - DNA, 77 bp, synthetic construct
  source 1..77: mol_type = other DNA
tggggactttccgctggggactttccgctggggactttccgctggggactttccgctggggactttccgcagctcct

SEQ ID NO: 12 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 13 - DNA, 106 bp, synthetic construct
  source 1..106: mol_type = other DNA
ctaaggtacgacggcgcctccagctaaaagagctaaaagaggtaagggtttaagggttggttggttggtggggtattaatctttaattatttttttttcaggcctg

SEQ ID NO: 14 - DNA, 1167 bp, synthetic construct
  source 1..1167: mol_type = other DNA
tggggactttccgctggggactttccgctggggactttccgctggggactttccgctggggactttccgcagctcctgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgactaaggtacgacggcgcctccagctaaaagagctaaaagaggtaagggtttaagggttggttggttggtggggtattaatctttaattatttttttttcaggcctg